(19) United States
(12) Patent Application Publication
(10) Pub. No.: US 2022/0347165 A1
(43) Pub. Date: Nov. 3, 2022

(54) EFFERVESCENT ORAL COMPOSITION

(71) Applicant: NICOVENTURES TRADING LIMITED, London (GB)

(72) Inventors: MICHAEL S. DANIEL, Winston Salem, NC (US); STEVEN LEE ALDERMAN, Lewisville, NC (US); ROSS JAY ODEN, Winston-Salem, NC (US)

(21) Appl. No.: 17/727,123

(22) Filed: Apr. 22, 2022

Related U.S. Application Data
(60) Provisional application No. 63/178,316, filed on Apr. 22, 2021.

(51) Int. Cl. A61K 31/465; A61K 9/46; A61K 9/20

(52) U.S. Cl. CPC A61K 31/465; A61K 9/0007; A61K 9/2018; A61K 9/2086; A61K 9/2059; A61K 9/2054

(57) ABSTRACT

The disclosure provides a multi-layered tablet configured for oral use, the tablet including a first layer including an effervescent composition and a second layer including a non-effervescent composition. The effervescent composition includes an effervescent material; one or more fillers in a total amount of at least about 30% by weight, the one or more fillers including at least one sugar alcohol; and at least one active ingredient. The non-effervescent composition includes one or more fillers in a total amount of at least about 30% by weight, the one or more fillers including at least one sugar alcohol; a binder; and a second active ingredient.

### CROSS-REFERENCE TO RELATED APPLICATIONS

This application claims priority to U.S. Provisional Application No. 63/178,316, filed on Apr. 22, 2021, and which is incorporated herein by reference in its entirety and for all purposes.

### FIELD OF THE DISCLOSURE

The present disclosure relates to products intended for human use. The products are configured for oral use and deliver substances such as flavors and/or active ingredients during use. Such products may include tobacco or a product derived from tobacco, or may be tobacco-free alternatives.

### BACKGROUND

Tobacco may be enjoyed in a so-called “smokeless” form. Particularly popular smokeless tobacco products are employed by inserting some form of processed tobacco or tobacco-containing formulation into the mouth of the user. Conventional formats for such smokeless tobacco products include moist snuff, snus, and chewing tobacco, which are typically formed almost entirely of particulate, granular, or shredded tobacco, and which are either portioned by the user or presented to the user in individual portions, such as in single-use pouches or sachets. Other traditional forms of smokeless products include compressed or agglomerated forms, such as plugs, tablets, or pellets. Alternative product formats, such as tobacco-containing gums and mixtures of tobacco with other plant materials, are also known. See for example, the types of smokeless tobacco formulations, ingredients, and processing methodologies set forth in U.S. Pat. No. 1,376,586 to Schwartz; U.S. Pat. No. 4,513,756 to Pittman et al.; U.S. Pat. No. 4,528,993 to Sensabaugh, Jr. et al.; 4,624,269 to Story et al.; U.S. Pat. No. 4,991,599 to Tibbetts; U.S. Pat. No. 4,987,907 to Townsend; U.S. Pat. No. 5,092,352 to Sprinkle, III et al.; U.S. Pat. No. 5,387,416 to White et al.; U.S. Pat. No. 6,668,839 to Williams; U.S. Pat. No. 6,834,654 to Williams; U.S. Pat. No. 6,953,040 to Atchley et al.; U.S. Pat. No. 7,032,601 to Atchley et al.; and 7,694,686 to Atchley et al.; US Pat. Pub. Nos. 2004/0020503 to Williams; 2005/0115580 to Quinter et al.; 2006/0191548 to Strickland et al.; 2007/0062549 to Holton, Jr. et al.; 2007/0186941 to Holton, Jr. et al.; 2007/0186942 to Strickland et al.; 2008/0029110 to Dube et al.; 2008/0029116 to Robinson et al.; 2008/0173317 to Robinson et al.; 2008/0209586 to Neilsen et al.; 2009/0065013 to Essen et al.; and 2010/0282267 to Atchley, as well as WO2004/095959 to Arnarp et al., each of which is incorporated herein by reference.

Smokeless tobacco product configurations that combine tobacco material with various binders and fillers have been proposed more recently, with example product formats including lozenges, pastilles, gels, extruded forms, melts, chews, gummies, and the like. See, for example, the types of products described in US Patent App. Pub. Nos. 2008/0196730 to Engstrom et al.; 2008/0305216 to Crawford et al.; 2009/0293889 to Kumar et al.; 2010/0291245 to Gao et al; 2011/0139164 to Mua et al.; 2012/0037175 to Cantrell et al.; 2012/0055494 to Hunt et al.; 2012/0138073 to Cantrell et al.; 2012/0138074 to Cantrell et al.; 2013/0074855 to Holton, Jr.; 2013/0074856 to Holton, Jr.; 2013/0152953 to Mua et al.; 2013/0274296 to Jackson et al.; 2015/0068545 to Moldoveanu et al.; 2015/0101627 to Marshall et al.; and 2015/0230515 to Lampe et al., each of which is incorporated herein by reference.

It would be desirable to provide products configured for oral use which may deliver active ingredients to the consumer in an enjoyable form.

### BRIEF SUMMARY

The present disclosure generally provides products configured for oral use. The products comprise an effervescent composition comprising an effervescent material, one or more fillers, and a flavoring agent, an active ingredient, or both. The products further comprise a non-effervescent composition comprising one or more fillers, a binder, and a flavoring agent, an active ingredient, or both. The present disclosure further generally provides multi-layered tablets comprising one or more layers comprising the effervescent composition, and one or more layers comprising the non-effervescent composition.

Accordingly, in one aspect is provided a multi-layered tablet configured for oral use, the tablet comprising a first layer comprising an effervescent composition and a second layer comprising a non-effervescent composition, wherein: the effervescent composition comprises an effervescent material capable of causing effervescence in the oral cavity; one or more fillers in a total amount of at least about 30% by weight, based on the total weight of the effervescent composition, wherein the one or more fillers include at least one sugar alcohol; and a first active ingredient; and the non-effervescent composition comprises one or more fillers in a total amount of at least about 30% by weight, based on the total weight of the non-effervescent composition, wherein the one or more fillers include at least one sugar alcohol; a binder; and a second active ingredient.

In some embodiments, the first and second active ingredients are independently selected from the group consisting of nicotine components, botanical materials, stimulants, amino acids, vitamins, cannabinoids, cannabimimetics, terpenes, nutraceuticals, pharmaceutical agents, and combinations thereof. In some embodiments, the first active ingredient comprises a nicotine component. In some embodiments, the first and second active ingredients comprise a nicotine component.

In some embodiments, the nicotine component is present in an amount from about 0.001 to about 10% by weight in each composition, calculated as the free base and based on the total weight of each composition.

In some embodiments, the one or more fillers comprise isomalt, polysaccharides, or a combination thereof. In some embodiments, the one or more fillers comprise isomalt, glucose, and starch-derived polysaccharides. In some embodiments, the one or more fillers comprise from about 60% to about 90% by weight of each of the effervescent and the non-effervescent compositions, based on the total weight of each composition.

In some embodiments, the particle size of the one or more fillers in the effervescent composition is less than about 35 microns.

In some embodiments, the effervescent composition, the non-effervescent composition, or both, further comprise one or more additives selected from the group consisting of flavorants, sweeteners, tobacco materials, taste modifiers, salts, binders, buffering agents, colorants, humectants, oral care additives, preservatives, disintegration aids, antioxidants, flow aids, compressibility aids, and combinations thereof.

In some embodiments, the particle size of any flavorants, sweeteners, taste modifiers, salts, binders, buffering agents, colorants, humectants, emulsifiers, oral care additives, preservatives, disintegration aids, antioxidants, flow aids, and compressibility aids which may be present in the effervescent composition is less than about 35 microns.

In some embodiments, the binder is a cellulose derivative. In some embodiments, the cellulose derivative is a cellulose ether. In some embodiments, the cellulose ether is hydroxypropylcellulose.

In some embodiments, the multi-layered tablet is substantially free of tobacco material.

In one embodiment, the effervescent material comprises an acid component and a base component, wherein the base component is a carbonate material, a bicarbonate material, or a mixture thereof.

In one embodiment, the acid component is a tricarboxylic acid, a dicarboxylic acid, or a combination thereof. In one embodiment, the acid component comprises a combination of a tricarboxylic acid and a dicarboxylic acid in a weight ratio of from about 2:1 to about 1:2. In one embodiment, the acid component is citric acid, tartaric acid, or a combination thereof. In one embodiment, the acid component is a combination of citric acid and tartaric acid in a ratio of from about 2:1 to about 1:2 by weight. In one embodiment, the acid component is a combination of citric acid and tartaric acid in a ratio of about 1:1 by weight.

In one embodiment, the base component is a bicarbonate material.

In one embodiment, the acid component is present in an amount of from about 10% to about 20% by weight, based on the total dry weight of the effervescent composition.

In one embodiment, the acid component and the base component are present in about a 1:1 molar ratio.

In one embodiment, the effervescent material has a particle size of less than about 180 microns.

In one embodiment, the effervescent material comprises a sugar material containing an entrapped gaseous component, such that release of the entrapped gaseous component occurs upon dissolution of the sugar material in the oral cavity. In one embodiment, the sugar material containing an entrapped gaseous component is in the form of a gasified sugar material in particulate form, the gasified sugar material particles being in admixture with the one or more fillers and active ingredient.

In one embodiment, the at least one active ingredient comprises one or more botanical materials, stimulants, amino acids, vitamins, cannabinoids, nutraceuticals, pharmaceutical agents, or combinations thereof.

In one embodiment, the active ingredient comprises caffeine.

In one embodiments, the effervescent composition comprises from about 10 to about 25 dry weight percent of the effervescent material; at least about 50 dry weight percent of the one or more fillers; and from about 1 to about 10 dry weight percent of caffeine. In one embodiment, the one or more fillers comprises mannitol, maltodextrin, isomalt, polysaccharides, or a combination thereof. In one embodiment, the one or more fillers comprises isomalt, glucose, and starch-derived polysaccharides. In one embodiment, the particle size of the filler is less than about 35 microns.

In one embodiment, the effervescent composition further comprises one or more additives selected from the group consisting of sweeteners, taste modifiers, salts, binders, buffering agents, colorants, humectants, oral care additives, preservatives, disintegration aids, antioxidants, flow aids, compressibility aids, and combinations thereof. In one embodiment, the particle size of any flavorants, sweeteners, taste modifiers, salts, binders, buffering agents, colorants, humectants, emulsifiers, oral care additives, preservatives, disintegration aids, antioxidants, flow aids, and compressibility aids which may be present is less than about 35 microns.

In another aspect is provided a composition for use in an oral product comprising at least two layered segments or portions, the composition comprising:

an effervescent portion comprising an effervescent material capable of causing effervescence in the oral cavity; one or more fillers in a total amount of at least about 30% by weight, based on the total weight of the effervescent portion, wherein the one or more fillers include at least one sugar alcohol; a first active ingredient; and

a non-effervescent portion comprising one or more fillers in a total amount of at least about 30% by weight, based on the total weight of the non-effervescent portion, wherein the one or more fillers include at least one sugar alcohol; a binder; and a second active ingredient,

wherein the oral product is in the form of a tablet.

The disclosure includes, without limitations, the following embodiments.

Embodiment 1: A multi-layered tablet configured for oral use, the tablet comprising a first layer comprising an effervescent composition and a second layer comprising a non-effervescent composition, wherein: the effervescent composition comprises an effervescent material capable of causing effervescence in the oral cavity; one or more fillers in a total amount of at least about 30% by weight, based on the total weight of the effervescent composition, wherein the one or more fillers include at least one sugar alcohol; a first active ingredient; and optionally, a lipid in an amount of at least about 20% by weight; and the non-effervescent composition comprises one or more fillers in a total amount of at least about 30% by weight, based on the total weight of the non-effervescent composition, wherein the one or more fillers include at least one sugar alcohol; a binder; and a second active ingredient.

Embodiment 2: The multi-layered tablet of embodiment 1, wherein the first and second active ingredients are independently selected from the group consisting of nicotine components, botanical materials, stimulants, amino acids, vitamins, cannabinoids, cannabimimetics, terpenes, nutraceuticals, pharmaceutical agents, and combinations thereof.

Embodiment 3: The multi-layered tablet of embodiment 1 or 2, wherein the first active ingredient comprises a nicotine component.

Embodiment 4: The multi-layered tablet of any one of embodiments 1-3, wherein the first and second active ingredients comprise a nicotine component.

Embodiment 5: The multi-layered tablet of any one of embodiments 1-4, wherein the nicotine component is present in an amount from about 0.001 to about 10% by weight in each composition, calculated as the free base and based on the total weight of each composition.

Embodiment 6: The multi-layered tablet of any one of embodiments 1-5, wherein the one or more fillers comprise isomalt, polysaccharides, or a combination thereof.

Embodiment 7: The multi-layered tablet of any one of embodiments 1-6, wherein the one or more fillers comprise isomalt, glucose, and starch-derived polysaccharides.

Embodiment 8: The multi-layered tablet of any one of embodiments 1-7, wherein the one or more fillers comprise from about 60% to about 90% by weight of each of the effervescent and the non-effervescent compositions, based on the total weight of each composition.

Embodiment 9: The multi-layered tablet of any one of embodiments 1-8, wherein the particle size of the one or more fillers in the effervescent composition is less than about 35 microns.

Embodiment 10: The multi-layered tablet of any one of embodiments 1-9, wherein the effervescent composition, the non-effervescent composition, or both, further comprise one or more additives selected from the group consisting of flavorants, sweeteners, tobacco materials, taste modifiers, salts, binders, buffering agents, colorants, humectants, oral care additives, preservatives, disintegration aids, antioxidants, flow aids, compressibility aids, and combinations thereof.

Embodiment 11: The multi-layered tablet of any one of embodiments 1-10, wherein the particle size of any flavorants, sweeteners, tobacco materials, taste modifiers, salts, binders, buffering agents, colorants, humectants, emulsifiers, oral care additives, preservatives, disintegration aids, antioxidants, flow aids, and compressibility aids which may be present in the effervescent composition is less than about 35 microns.

Embodiment 12: The multi-layered tablet of any one of embodiments 1-11, wherein the binder is a cellulose derivative.

Embodiment 13: The multi-layered tablet of any one of embodiments 1-12, wherein the cellulose derivative is a cellulose ether.

Embodiment 14: The multi-layered tablet of any one of embodiments 1-13, wherein the cellulose ether is hydroxypropylcellulose.

Embodiment 15: The multi-layered tablet of any one of embodiments 1-14, wherein the multi-layered tablet is substantially free of tobacco material.

Embodiment 16: A composition for use in an oral product comprising at least two layered portions, the composition comprising:

an effervescent portion comprising an effervescent material capable of causing effervescence in the oral cavity; one or more fillers in a total amount of at least about 30% by weight, based on the total weight of the effervescent portion, wherein the one or more fillers include at least one sugar alcohol; a first active ingredient; and

a non-effervescent portion comprising one or more fillers in a total amount of at least about 30% by weight, based on the total weight of the non-effervescent portion, wherein the one or more fillers include at least one sugar alcohol; a binder; and a second active ingredient,

wherein the oral product is in the form of a tablet.

These and other features, aspects, and advantages of the disclosure will be apparent from a reading of the following detailed description together with the accompanying drawing, which are briefly described below. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise.

### BRIEF DESCRIPTION OF THE DRAWINGS

Having thus described aspects of the disclosure in the foregoing general terms, reference will now be made to the accompanying drawing, which is not necessarily drawn to scale. The drawing is exemplary only, and should not be construed as limiting the disclosure.

FIG. 1A is a perspective view of an example embodiment of an effervescent composition of the present disclosure in the form of a tablet having a diameter and a thickness;

FIG. 1B is a perspective view of another example embodiment of an effervescent composition of the present disclosure in the form of a tablet having a width, length, and thickness;

FIG. 2A is a perspective view of an example embodiment of an effervescent composition of the present disclosure in the form of a layered tablet having a top and bottom layer, wherein at least one of the layers comprises the effervescent composition;

FIG. 2B is a perspective view of an example embodiment of an effervescent composition of the present disclosure in the form of a layered tablet having an inner and an outer layer, wherein at least one of the layers comprises the effervescent composition; and

FIG. 3 is a perspective view of an example embodiment of the present disclosure in the form of a two-layered tablet having an effervescent layer and a non-effervescent layer.

### DETAILED DESCRIPTION

The present disclosure provides an effervescent composition adapted for oral use, comprising an effervescent material capable of causing effervescence in the oral cavity. Surprisingly, it has been found according to the present disclosure that the presence of an effervescent effect in the oral cavity reduces the perception of bitterness associated with certain active ingredients (e.g., caffeine or nicotine) which may be present in a composition adapted for oral use.

The present disclosure will now be described more fully hereinafter with reference to example embodiments thereof. These example embodiments are described so that this disclosure will be thorough and complete, and will fully convey the scope of the disclosure to those skilled in the art. Indeed, the disclosure may be embodied in many different forms and should not be construed as limited to the embodiments set forth herein; rather, these embodiments are provided so that this disclosure will satisfy applicable legal requirements.

As used in this specification and the claims, the singular forms “a,” “an,” and “the” include plural referents unless the context clearly dictates otherwise. The term “about” used throughout this specification is used to describe and account for small fluctuations. For example, the term “about” can refer to less than or equal to ±10%, such as less than or equal to ±5%, less than or equal to ±2%, less than or equal to ±1%, less than or equal to ±0.5%, less than or equal to ±0.2%, less than or equal to ±0.1% or less than or equal to ±0.05%. All numeric values herein are modified by the term “about,” whether or not explicitly indicated. A value modified by the term “about” of course includes the specific value. For instance, “about 5.0” must include 5.0.

Reference to “dry weight percent” or “dry weight basis” refers to weight on the basis of dry ingredients (i.e., all ingredients except water). Reference to “wet weight” refers to the weight of the effervescent composition including water. Unless otherwise indicated, reference to “weight percent” of a composition reflects the total wet weight of the composition (i.e., including water).

### Effervescent and Non-Effervescent Compositions

In one aspect is provided a multi-layered tablet configured for oral use, the tablet comprising at least one layer or a first layer comprising an effervescent composition as described herein, and at least one or a second layer comprising a non-effervescent composition as described herein. The effervescent composition comprises an effervescent material capable of causing effervescence in the oral cavity; one or more fillers; a flavoring agent, an active ingredient, or both; and, optionally, a lipid. The non-effervescent composition comprises one or more fillers; a binder; and a flavoring agent, an active ingredient, or both; and, optionally, a lipid.

Each of the components may be selected individually for the effervescent and non-effervescent compositions. Accordingly, the individual components (e.g., fillers, binders, flavoring agents, active ingredients, and lipid) present in the non-effervescent composition may be the same or may be different from those present in the effervescent composition, and may be present in the same general amount ranges provided herein. In some embodiments, each of the one or more fillers, one or more sweeteners, flavoring agent, and/or active ingredient are the same in each composition.

The relative amounts of the various components within the effervescent and non-effervescent compositions may vary, and typically are selected so as to provide the desired sensory and performance characteristics to the overall product. The example individual components of the effervescent and non-effervescent compositions are described herein below.

### Effervescent Material

As used herein, the term “effervescent material” refers to a material which, upon contact with moisture (e.g., saliva in the oral cavity of a consumer of the effervescent composition as disclosed herein), releases bubbles of a gas resulting in a foaming or fizzing action.

In some embodiments, the effervescent material is a combination of two or more components capable of reacting, typically in an aqueous environment, to produce a gas. The resulting gas is typically carbon dioxide, although it is possible to use reactive couples that produce other gases that are safe for human consumption, such as oxygen. The presence of the effervescent materials adds distinctive organoleptic properties to the product, particularly in terms of taste and mouthfeel. In particular, the presence of effervescent materials masks or alters the perception of bitterness, for example, of an active ingredient present in the composition. It also allows for substantially complete dissolution and oral delivery of e.g., active ingredient(s) during oral use as opposed to a consumer needing to swallow a composition comprising the same active ingredient(s) to effect delivery of the active ingredient(s). Advantageously, this effervescence further eliminates the need for long-acting excipients to achieve dissolution and oral delivery.

The use of effervescent materials is described, for example, in U.S. Pat. No. 4,639,368 to Niazi et al.; U.S. Pat. No. 5,178,878 to Wehling et al.; U.S. Pat. No. 5,223,264 to Wehling et al.; U.S. Pat. No. 6,974,590 to Pather et al.; and U.S. Pat. No. 7,381,667 to Bergquist et al., as well as U.S. Pat. Pub. Nos. 2006/0191548 to Strickland et al.; 2009/0025741 to Crawford et al; 2010/0018539 to Brinkley et al.; and 2010/0170522 to Sun et al.; and PCT WO 97/06786 to Johnson et al., all of which are incorporated by reference herein.

In certain embodiments, the effervescent material can include an acid/base pair that provides the effervescent effect of the composition. See, for example, the use of acids and bases in effervescent compositions described in U.S. Pat. Pub. No. 2012/0055494 to Hunt et al., which is incorporated by reference herein.

In one embodiment, the effervescent material is a reactive couple comprising at least one acid component (an acid, an anhydride, or an acid salt) and at least one base capable of reacting with the acid component to release carbon dioxide. Multiple acids and multiple bases can be combined in the same product to produce the desired reaction.

### Acid Component

In certain embodiments, the acid component of the effervescent material is selected from carboxylic acids having about 2 to about 12 carbon atoms (e.g., C₂-C₁₀ or C₂-C₈ or C₂-C₆ carboxylic acids), wherein the carboxylic acids are monoprotic or polyprotic (e.g., dicarboxylic acids or tricarboxylic acids). Exemplary organic acids include citric acid, malic acid, tartaric acid, succinic acid, adipic acid, fumaric acid, gluconic acid, and combinations thereof. Example acid salts include acidic sodium salts, acidic calcium salts, dihydrogen phosphate salts, and disodium dihydrogen pyrophosphate salts. In some embodiments, the acid component is citric acid or tartaric acid.

In some embodiments, a combination of acids is utilized where at least one acid is a polyprotic acid, such as a dicarboxylic acid (tartaric acid) or a tricarboxylic acid (e.g., citric acid). Combinations of a dicarboxylic acid and a tricarboxylic acid are also suitable for use in the effervescent material. In some embodiments, the acid component comprises a combination of a tricarboxylic acid and a dicarboxylic acid in a weight ratio of from about 2:1 to about 1:2, for example, from about 2:1, about 1.5:1, or about 1:1, to about 1:1.5, or about 1:2. In some embodiments, the acid component is a combination of citric acid and tartaric acid in a ratio of from about 2:1 to about 1:2 by weight. In specific embodiments, the acid component is a combination of citric acid and tartaric acid in a 1:1 ratio by weight.

The amount of acid component of the effervescent material in the composition can vary, but is typically from about 1 to about 25 dry weight percent, such as about 5 to about 20 dry weight percent, or about 10 to about 18 dry weight percent (e.g., about 10, about 11, about 12, about 13, about 14, about 15, about 16, about 17, or about 18 dry weight percent). Where three or more acids are utilized, each acid is typically present in an amount of about 10 to about 35 dry weight percent, based on the total weight of the acid component. In one embodiment, the acid component is 5% by weight of citric acid and 5% by weight tartaric acid. The acid component, e.g., citric and/or tartaric acid particles, may be encapsulated or coated.

### Base Component

Examples of suitable base components include carbonate and bicarbonate materials, particularly alkali metal or alkaline earth metal salts thereof. Carbonate and bicarbonate base materials capable of use in the present invention include sodium carbonate, sodium bicarbonate, potassium carbonate, potassium bicarbonate, magnesium carbonate, calcium carbonate, sodium sesquicarbonate, sodium glycine carbonate, lysine carbonate, and arginine carbonate. In some embodiments, the base component is sodium bicarbonate. The base component, e.g., sodium bicarbonate particles may be encapsulated or coated. Encapsulated sodium bicarbonate is available from, for example Watson (301 Heffernan Drive West Haven, Conn. 06516) or Clabber Girl Corporation (900 Wabash Ave, Terre Haute, IN 47807).

In some embodiments, a combination of carbonate salts and bicarbonate components may be used. Bicarbonate materials, while highly reactive in effervescent reactions, are not efficient buffering agents in certain desirable pH ranges. Thus, in certain embodiments utilizing both a bicarbonate and carbonate material, it is advantageous to stoichiometrically match the bicarbonate amount to the acid component of the effervescent material and use a carbonate material as the main buffering agent. In this manner, although the carbonate material would be expected to participate in the effervescent reaction to a limited degree, the bicarbonate material is present in an amount sufficient to fully react with the available acid component and the carbonate material is present in an amount sufficient to provide the desired pH range.

The amount of the base component (e.g., carbonate or bicarbonate materials) of the effervescent material in the effervescent composition can vary, but is typically about 4 to about 30 dry weight percent, for example, from about 5 to about 25 dry weight percent, about 8 to about 20 dry weight percent, or about 6 to about 12 dry weight percent (e.g., about 4, about 6, about 8, about 10, about 12, about 14, about 16, about 18, or about 20 dry weight percent). In certain embodiments, the effervescent composition may include both a carbonate component and a bicarbonate component. For such embodiments, the amount of carbonate material can vary, but is typically about 3 to about 20 dry weight percent, such as about 5 to about 15 dry weight percent, or about 8 to about 15 dry weight percent (e.g., about 8, about 9, about 10, about 11, about 12, about 13, or about 14 dry weight percent). For such embodiments, the amount of bicarbonate material can vary, but is typically about 3 to about 20 dry weight percent, often about 5 to about 15 dry weight percent, and most often about 8 to about 15 dry weight percent (e.g., about 8, about 9, about 10, about 11, about 12, about 13, or about 14 dry weight percent). In some embodiments, the base component is sodium bicarbonate, present in an amount by weight of about 12%, based on the total weight of the effervescent composition.

In certain embodiments, it is desirable for the reaction between the acid and base component to proceed completely. To ensure this result, the relevant amount of acid and base can be adjusted so that the necessary equivalent amounts are present. In some embodiments, the acid component and the base component are present in about a 1:1 molar ratio. For example, if a dicarboxylic acid is used, then either a di-reactive base can be used in roughly equivalent amount or a mono-reactive base could be used at a level roughly twice that of the acid. Likewise, if a tricarboxylic acid is used, then either a tri-reactive base can be used in roughly equivalent amount or a mono-reactive base could be used at a level roughly thrice that of the acid. Alternatively, an excess amount of either acid or base can be used, particularly where the acid or base is intended to provide an independent effect on the organoleptic properties of the effervescent composition beyond simply providing effervescence.

The amount of total effervescent material (i.e., all reactive materials that produce the gaseous product) in the effervescent composition can vary. The amount of such material should be sufficient to enable the effervescent composition to effervesce when placed in the oral cavity. The amount of effervescent material is typically about 5 to about 50 dry weight percent, for example, from about 8 to about 30 dry weight percent, about 10 to about 25 dry weight percent (e.g., about 10, about 12, about 14, about 16, about 18, about 20, or about 22 dry weight percent), based on the total weight of the effervescent composition. The amount of effervescent material in some embodiments can be characterized as at least about 10 dry weight percent, or at least about 15 dry weight percent, or at least about 20 dry weight percent, or at least about 25 dry weight percent. The amount of effervescent material in some embodiments can be characterized as no more than about 50 dry weight percent, no more than about 40 dry weight percent, no more than about 35 dry weight percent, no more than about 30 dry weight percent, or no more than about 20 dry weight percent.

The amount of gas (e.g., carbon dioxide) that evolves from the effervescence reaction in the effervescent composition can vary, and depends in part on the desired sensory characteristics of the effervescent composition. The amount of effervescent material can be selected to achieve the desired level of carbon dioxide release. One method for measuring the amount of carbon dioxide released from a given quantity of effervescent composition involves the following steps: (1) pipetting 1 ml of water to a vial; (2) capping the vial; (3) pre-weighing the capped vial using, for example, a Mettler Model AE 163 balance or equivalent analytical balance readable to 0.0001 g; (4) reweighing the capped vial along with the effervescent composition to be tested; (5) add the effervescent composition to the water in the vial and cap the vial loosely (tighten cap until barely tight and then loosen cap slightly); (6) after about thirty minutes, vortexing the vials for 3-4 seconds using a vortex mixer such as a Fisher Scientific Touch Mixer Model 232 or equivalent; (8) loosening cap to release trapped gas and then again capping vial loosely; (9) after about one hour, repeating Steps 7 and 8 and reweighing vial; and (10) after about 1.5 hours, repeat Steps 7 and 8 and reweighing vial. The amount of carbon dioxide evolved from the effervescent composition is the difference in weight between Step 4 to Step 10.

In the above test, the intent is to use enough water in the vial to initiate the reaction between acid and base, but not so much that an appreciable amount of carbon dioxide remains dissolved in the water. Vortexing the sample agitates the liquid to overcome supersaturation of the water with carbon dioxide. The vials are loosely capped to allow carbon dioxide to escape without allowing water to evaporate. Carbon dioxide is heavier than air, so weights at different time points are taken to make sure that the carbon dioxide has diffused out of the head space of the vial. The last two vial weights should agree within about 1.5 mg.

The amount of evolved carbon dioxide from an effervescent composition of the invention can be expressed as a ratio of weight of carbon dioxide evolved to total effervescent composition weight. In certain embodiments, this ratio can be from about 10 μg carbon dioxide/mg of effervescent composition to about 120 μg carbon dioxide/mg of effervescent composition, from about 10 μg carbon dioxide/mg to about 60 μg carbon dioxide/mg, or from about 10 μg carbon dioxide/mg to about 30 μg carbon dioxide/mg. In certain embodiments, the amount of evolved carbon dioxide can be characterized as at least about 10 μg carbon dioxide/mg of effervescent composition, or at least about 15 μg carbon dioxide/mg of effervescent composition.

### Particulate Material

In some embodiments, any one or more components of the effervescent composition disclosed herein can be described as a particulate material. As used herein, the term “particulate” refers to a material in the form of a plurality of individual particles, some of which can be in the form of an agglomerate of multiple particles, wherein the particles have an average length to width ratio less than 2:1, such as less than 1.5:1, such as about 1:1. In various embodiments, the particles of a particulate material can be described as substantially spherical or granular.

The particle size of a particulate material may be measured by sieve analysis. As the skilled person will readily appreciate, sieve analysis (otherwise known as a gradation test) is a method used to measure the particle size distribution of a particulate material. Typically, sieve analysis involves a nested column of sieves which comprise screens, preferably in the form of wire mesh cloths. A pre-weighed sample may be introduced into the top or uppermost sieve in the column, which has the largest screen openings or mesh size (i.e. the largest pore diameter of the sieve). Each lower sieve in the column has progressively smaller screen openings or mesh sizes than the sieve above. Typically, at the base of the column of sieves is a receiver portion to collect any particles having a particle size smaller than the screen opening size or mesh size of the bottom or lowermost sieve in the column (which has the smallest screen opening or mesh size).

In some embodiments, the column of sieves may be placed on or in a mechanical agitator. The agitator causes the vibration of each of the sieves in the column. The mechanical agitator may be activated for a pre determined period of time in order to ensure that all particles are collected in the correct sieve. In some embodiments, the column of sieves is agitated for a period of time from 0.5 minutes to 10 minutes, such as from 1 minute to 10 minutes, such as from 1 minute to 5 minutes, such as for approximately 3 minutes. Once the agitation of the sieves in the column is complete, the material collected on each sieve is weighed. The weight of each sample on each sieve may then be divided by the total weight in order to obtain a percentage of the mass retained on each sieve. As the skilled person will readily appreciate, the screen opening sizes or mesh sizes for each sieve in the column used for sieve analysis may be selected based on the granularity or known maximum/minimum particle sizes of the sample to be analysed. In some embodiments, a column of sieves may be used for sieve analysis, wherein the column comprises from 2 to 20 sieves, such as from 5 to 15 sieves. In some embodiments, a column of sieves may be used for sieve analysis, wherein the column comprises 10 sieves. In some embodiments, the largest screen opening or mesh sizes of the sieves used for sieve analysis may be 1000 μm, such as 500 μm, such as 400 μm, or such as 300 μm.

In some embodiments, any material referenced herein (e.g., filler, effervescent material, active ingredient, and the overall effervescent composition, as well any materials within the non-effervescent composition) has particles with a particle size as measured by sieve analysis of no greater than about 1000 μm, such as no greater than about 500 μm, such as no greater than about 400 μm, such as no greater than about 350 μm, such as no greater than about 300 μm, such as no greater than about 250 μm, such as no greater than about 200 μm, such as no greater than about 150 μm, such as no greater than about 100 μm, such as no greater than about 50 μm, such as no greater than about 40 μm, such as no greater than about 30 μm. In some embodiments, at least 60% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm, such as no greater than about 250 μm, such as no greater than about 200 μm, such as no greater than about 150 μm, such as no greater than about 100 μm, such as no greater than about 50 μm, such as no greater than about 40 μm, such as no greater than about 30 μm. In some embodiments, at least 70% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm, such as no greater than about 250 μm, such as no greater than about 200 μm, such as no greater than about 150 μm, such as no greater than about 100 μm, such as no greater than about 50 μm, such as no greater than about 40 μm, such as no greater than about 30 μm. In some embodiments, at least 80% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm, such as no greater than about 250 μm, such as no greater than about 200 μm, such as no greater than about 150 μm, such as no greater than about 100 μm, such as no greater than about 50 μm, such as no greater than about 40 μm, such as no greater than about 30 μm. In some embodiments, at least 90% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm, such as no greater than about 250 μm, such as no greater than about 200 μm, such as no greater than about 150 μm, such as no greater than about 100 μm, such as no greater than about 50 μm, such as no greater than about 40 μm, such as no greater than about 30 μm. In some embodiments, at least 95% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm, such as no greater than about 250 μm, such as no greater than about 200 μm, such as no greater than about 150 μm, such as no greater than about 100 μm, such as no greater than about 50 μm, such as no greater than about 40 μm, such as no greater than about 30 μm. In some embodiments, at least 99% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm, such as no greater than about 250 μm, such as no greater than about 200 μm, such as no greater than about 150 μm, such as no greater than about 100 μm, such as no greater than about 50 μm, such as no greater than about 40 μm, such as no greater than about 30 μm. In some embodiments, approximately 100% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm, such as no greater than about 250 μm, such as no greater than about 200 μm, such as no greater than about 150 μm, such as no greater than about 100 μm, such as no greater than about 50 μm, such as no greater than about 40 μm, such as no greater than about 30 μm.

In some embodiments, at least 50% by weight, such as at least 60% by weight, such as at least 70% by weight, such as at least 80% by weight, such as at least 90% by weight, such as at least 95% by weight, such as at least 99% by weight of the particles of any particulate material referenced herein have a particle size as measured by sieve analysis of from about 0.01 μm to about 1000 μm, such as from about 0.05 μm to about 750 μm, such as from about 0.1 μm to about 500 μm, such as from about 0.25 μm to about 500 μm. In some embodiments, at least 50% by weight, such as at least 60% by weight, such as at least 70% by weight, such as at least 80% by weight, such as at least 90% by weight, such as at least 95% by weight, such as at least 99% by weight of the particles of any particulate material referenced herein have a particle size as measured by sieve analysis of from about 10 μm to about 400 μm, such as from about 50 μm to about 350 μm, such as from about 100 μm to about 350 μm, such as from about 200 μm to about 300 μm.

In some embodiments, the effervescent material has a particle size as measured by sieve analysis of less than about 180 μm, such as less than about 150, less than about 120, less than about 80, less than about 60 μm, or less than about 40 μm. In some embodiments, the effervescent material has a particle size as measured by sieve analysis from about 30 to about 180 μm, such as from about 30 to about 40 μm, or from about 50 to about 60 μm. Without wishing to be bound by theory, it is believed that effervescent material particle sizes greater than about 180 μm contribute an unpleasantly rough texture to the effervescent composition. Conversely, effervescent material particle sizes smaller than about 180 μm produce, in some embodiments, a faster onset of effervescence, and provide a smoother texture.

In some embodiments, the particle size as measured by sieve analysis of the remaining effervescent composition components, and/or components of the non-effervescent composition as described herein below (e.g., filler, active ingredients, flavorants, sweeteners, taste modifiers, salts, binders, buffering agents, colorants, humectants, oral care additives, preservatives, disintegration aids, antioxidants, flow aids, compressibility aids) is less than about 50 μm, such as less than about 40 μm, less than about 35 μm, or less than about 30 μm. In some embodiments, the particle size as measured by sieve analysis of the remaining effervescent or non-effervescent composition components which may be present is from about 25, about 30, or about 35, to about 40, about 45, or about 50 μm. In some embodiments, the particle size as measured by sieve analysis of the remaining effervescent or non-effervescent composition components which may be present is from about 25 to about 35 μm. In some embodiments, the particle size as measured by sieve analysis of the remaining effervescent or non-effervescent composition components which may be present is less than about 32 μm.

### Gasified Material

In certain embodiments, the effervescence may be produced by release of entrapped gas rather than by chemical reaction. Accordingly, in some embodiments, the effervescent material comprises a sugar material containing an entrapped gaseous component, such that release of the entrapped gaseous component occurs upon dissolution of the sugar material in the oral cavity. In some embodiments, the sugar material containing an entrapped gaseous component is in the form of a gasified sugar material in particulate form, the gasified sugar material particles being in admixture with the filler and active ingredient. As used herein, “gasified sugar material” refers to a sugar material containing an entrapped gaseous component capable of release upon dissolution of the sugar material in the oral cavity. The gasified sugar material is typically provided in solid form (e.g., granular or particulate form). The average particle size of the gasified sugar material can vary, but is typically about 50 to about 800 microns, more often about 100 to about 600 microns, and most often about 125 to about 500 microns. The gasified sugar material is advantageously maintained in a very dry state to avoid premature effervescence during handling or storage. For example, the gasified sugar material will typically comprise less than about 5% water by weight, less than about 3% water by weight, less than about 2% water by weight, or less than about 1% water by weight.

Commercially available examples of gasified sugar material are sold under the brand name Carbonated Crystals™ by Raven Manufacturing, LLC of Neenah, Wis. Exemplary methods for forming gasified sugar materials are set forth in U.S. Pat. No. 4,289,794 to Kleiner et al.; U.S. Pat. No. 5,165,951 to Gallart et al., and U.S. Pat. No. 5,439,698 to Ahn et al, all of which are incorporated by reference herein. Typical manufacturing processes involve introducing a gaseous component (e.g., carbon dioxide) under pressure (e.g., 50 to 650 psig) to the sugar material while the sugar is in melted form.

The amount of gasified sugar material in the effervescent composition can vary, and will depend in part on the desired organoleptic properties of the effervescent composition. Typically, the amount of gasified sugar material (including the total weight of sugar materials and entrapped gas) is in the range of about 10 to about 90 dry weight percent, based on the total weight of the effervescent composition, such as about 20 to about 60 dry weight percent, or about 30 to about 50 dry weight percent.

The sugar component of the gasified sugar material can be any of a variety of monosaccharides (e.g., glucose, fructose, galactose), disaccharides (e.g., sucrose, lactose, maltose), trisaccharides, or oligosaccharides. Although sucrose or other nutritive sweeteners can be used as the sugar material, the effervescent composition of the disclosure can also be prepared as a sugar-free product, meaning the gasified sugar material can be characterized as a sugar substitute. “Sugar-free” as used herein is intended to include products having less than about 1/15th sugar by weight, or less than about 1/10th sugar by weight.

### Filler

Compositions as described herein (both effervescent and non-effervescent) include one or more fillers. Such fillers may fulfill multiple functions, such as enhancing certain organoleptic properties such as texture and mouthfeel, enhancing cohesiveness or compressibility of the product, and the like. Generally, the fillers are porous particulate materials and are cellulose-based. For example, suitable fillers are any non-tobacco plant material or derivative thereof, including cellulose materials derived from such sources. Examples of cellulosic non-tobacco plant material include cereal grains (e.g., maize, oat, barley, rye, buckwheat, and the like), sugar beet (e.g., FIBREX® brand filler available from International Fiber Corporation), bran fiber, and mixtures thereof. Non-limiting examples of derivatives of non-tobacco plant material include starches (e.g., from potato, wheat, rice, corn), natural cellulose, and modified cellulosic materials.

“Starch” as used herein may refer to pure starch from any source, modified starch, or starch derivatives. Starch is present, typically in granular form, in almost all green plants and in various types of plant tissues and organs (e.g., seeds, leaves, rhizomes, roots, tubers, shoots, fruits, grains, and stems). Starch can vary in composition, as well as in granular shape and size. Often, starch from different sources has different chemical and physical characteristics. A specific starch can be selected for inclusion in the effervescent composition based on the ability of the starch material to impart a specific organoleptic property to the effervescent composition. Starches derived from various sources can be used. For example, major sources of starch include cereal grains (e.g., rice, wheat, and maize) and root vegetables (e.g., potatoes and cassava). Other examples of sources of starch include acorns, arrowroot, arracacha, bananas, barley, beans (e.g., favas, lentils, mung beans, peas, chickpeas), breadfruit, buckwheat, canna, chestnuts, colacasia, katakuri, kudzu, malanga, millet, oats, oca, Polynesian arrowroot, sago, sorghum, sweet potato, quinoa, rye, tapioca, taro, tobacco, water chestnuts, and yams. Certain starches are modified starches. A modified starch has undergone one or more structural modifications, often designed to alter its high heat properties.

Some starches have been developed by genetic modifications, and are considered to be “genetically modified” starches. Other starches are obtained and subsequently modified by chemical, enzymatic, or physical means. For example, modified starches can be starches that have been subjected to chemical reactions, such as esterification, etherification, oxidation, depolymerization (thinning) by acid catalysis or oxidation in the presence of base, bleaching, transglycosylation and depolymerization (e.g., dextrinization in the presence of a catalyst), cross-linking, acetylation, hydroxypropylation, and/or partial hydrolysis. Enzymatic treatment includes subjecting native starches to enzyme isolates or concentrates, microbial enzymes, and/or enzymes native to plant materials, e.g., amylase present in corn kernels to modify corn starch. Other starches are modified by heat treatments, such as pregelatinization, dextrinization, and/or cold water swelling processes. Certain modified starches include monostarch phosphate, distarch glycerol, distarch phosphate esterified with sodium trimetaphosphate, phosphate distarch phosphate, acetylated distarch phosphate, starch acetate esterified with acetic anhydride, starch acetate esterified with vinyl acetate, acetylated distarch adipate, acetylated distarch glycerol, hydroxypropyl starch, hydroxypropyl distarch glycerol, and starch sodium octenyl succinate.

Combinations of fillers can also be used. For example, in some embodiments, the one or more fillers comprise a mixture of glucose and starch-derived polysaccharides. One such suitable mixture of glucose and starch-derived polysaccharides is EMDEX®, available from JRS PHARMA LP, USA, 2981 Route 22, Patterson, NY 12563-2359.

In some embodiments, the filler comprises a cellulose material or a cellulose derivative, such as microcrystalline cellulose (“mcc”). The mcc may be synthetic or semi-synthetic, or it may be obtained entirely from natural celluloses. The mcc may be selected from the group consisting of AVICEL® grades PH-100, PH-102, PH-103, PH-105, PH-112, PH-113, PH-200, PH-300, PH-302, VWACEL® grades 101, 102, 12, 20 and EMOCEL® grades 50M and 90M, and the like, and mixtures thereof.

Additional examples of potential fillers include maltodextrin, dextrose, calcium carbonate, calcium phosphate, lactose, and sugar alcohols. In some embodiments, the one or more fillers includes at least one sugar alcohol. Sugar alcohols are polyols derived from monosaccharides or disaccharides that have a partially or fully hydrogenated form. Sugar alcohols have, for example, about 4 to about 20 carbon atoms and include erythritol, arabitol, ribitol, isomalt, maltitol, dulcitol, iditol, mannitol, xylitol, lactitol, sorbitol, and combinations thereof (e.g., hydrogenated starch hydrolysates). In some embodiments, the one or more fillers comprise at least one of mannitol, maltodextrin, isomalt, and starch-derived polysaccharides. In some embodiments, one or more fillers comprise mannitol. In some embodiments, the one or more fillers comprise isomalt. In some embodiments, the one or more fillers is a combination of mannitol and maltodextrin. In some embodiments, the one or more fillers comprises isomalt, glucose, and starch-derived polysaccharides. In some embodiments, the one or more fillers comprises EMDEX®. In some embodiments, the one or more fillers comprises a combination of isomalt and EMDEX®.

The amount of filler can vary, but is typically greater than about 25%, and up to about 85% of the individual effervescent and non-effervescent compositions by weight, based on the total weight of each individual composition. A typical range of filler within the compositions can be from about 25 to about 85% by total weight of each composition, for example, from about 25, about 30, about 35, about 40, about 45, or about 50%, to about 55, about 60%, about 65, about 70%, about 75%, or about 80% by weight (e.g., about 20 to about 50%, or about 25 to about 45% by weight). In certain embodiments, the amount of filler is at least about 30% by weight, such as at least about 35%, or at least about 35%, or at least about 40%, or at least about 45%, or at least about 50%, based on the total weight of each individual composition. In some embodiments, the one or more fillers are present in a total amount of up to about 50% by weight, based on the total weight of each individual composition. In some embodiments, the one or more fillers are present in a total amount of at least about 30% by weight, based on the total weight of each individual composition. In particular embodiments, the one or more fillers in the effervescent composition includes at least one sugar alcohol. In particular embodiments, the one or more fillers in the effervescent composition comprises from about 25 to about 35% of isomalt and from about 30 to about 45% of EMDEX®. In particular embodiments, the one or more fillers in the non-effervescent composition includes at least one sugar alcohol. In particular embodiments, the one or more fillers in the non-effervescent composition comprises from about 20 to about 30% of isomalt and from about 40 to about 55% of EMDEX®.

### Lipid

In some embodiments, the effervescent composition, the non-effervescent composition, or both comprise a lipid. Such compositions may, in some embodiments, be described as “meltable” or “melting” compositions. The lipid is typically a fat, oil, or wax substance derived from animal or plant material (e.g., plant-derived fats), and typically comprises mostly triglycerides along with lesser amounts of free fatty acids and mono- or diglycerides. In certain embodiments, the lipid is a solid or semi-solid at room temperature (i.e., 25° C.) and capable of at least partially liquefying when subjected to the temperature of the oral cavity of the user (i.e., “melting”). Example plant-derived fats are comprised primarily of saturated or unsaturated fatty acid chains (most of which are bound within triglyceride structures) having a carbon length of about 10 to about 26 carbon atoms, or about 14 to about 20 carbon atoms, or about 14 to about 18 carbon atoms.

In some embodiments, the lipid comprises an oil and, in particular, a food grade oil. Such oils include, but are not limited to, vegetable oils (e.g., acai oil, almond oil, amaranth oil, apricot oil, apple seed oil, argan oil, avocado oil, babassu oil, beech nut oil, ben oil, bitter gourd oil, black seed oil, blackcurrant seed oil, borage seed oil, borneo tallow nut oil, bottle gourd oil, brazil nut oil, buffalo gourd oil, butternut squash seed oil, cape chestnut oil, canola oil, carob cashew oil, cocoa butter, cocklebur oil, coconut oil, corn oil, cothune oil, coriander seed oil, cottonseed oil, date seed oil, dika oil, egus seed oil, evening primrose oil, false flax oil, flaxseed oil, grape seed oil, grapefruit seed oil, hazelnut oil, hemp oil, kapok seed oil, kenaf seed oil, lallemantia oil, lemon oil, linseed oil, macadamia oil, mafura oil, manila oil, meadowfoam seed oil, mongongo nut oil, mustard oil, niger seed oil, nutmeg butter, okra seed oil, olive oil, orange oil, palm oil, palm stearin, papaya seed oil, peanut oil, pecan oil, perilla seed oil, persimmon seed oil, pequi oil, pili nut oil, pine nut oil, pistachio oil, pomegranate seed oil, poppyseed oil, pracaxi oil, prune kernel oil, pumpkin seed oil, quinoa oil, ramtil oil, rapeseed oil, rice bran oil, royle oil, sacha inchi oil, safflower oil, sapote oil, seje oil, sesame oil, shea butter, soybean oil, sunflower oil, taramira oil, tea seed oil, thistle oil, tigernut oil, tobacco seed oil, tomato seed oil, walnut oil, watermelon seed oil, wheat germ oil, and combinations thereof), animal oils (e.g., cattle fat, buffalo fat, sheep fat, goat fat, pig fat, lard, camel fat, tallow, liquid margarine, fish oil, fish liver oil, whale oil, seal oil, and combinations thereof), and mineral oils.

In certain embodiments, the plant-derived fats of the present disclosure include palm oil, palm kernel oil, soybean oil, cottonseed oil, and mixtures thereof. In one embodiment, the lipid is a blend of palm oil and palm kernel oil. The lipid can be, for example, hydrogenated, partially hydrogenated, or non-hydrogenated. Example embodiments of lipids can be purchased under the brand names CEBES®, CISAO®, or CONFAO®, available from AarhusKarlshamn USA Inc, and under the brand name Paramount, available from Bunge Loders Croklaan.

The melting point of the lipid is typically about 29° C. or above, such as about 29° C. to about 49° C., or about 36° C. to about 45° C., or about 38° C. to about 41° C. In some embodiments, use of lipids with a melting point of less than about 36° C. is not advantageous due to possible melting during product storage or handling. One test for determining the melting point of lipids is the Mettler dropping point method (ASTM D3954-15, Standard Test Method for Dropping Point of Waxes, ASTM International, West Conshohocken, Pa., 2015, www.astm.org.).

The amount of lipid within the effervescent composition, the non-effervescent composition, or both may vary. In certain embodiments, the amount of lipid is at least about 10 percent, at least about 20 percent, or at least about 30 percent, on a dry weight basis of the individual composition. In certain embodiments, the amount of lipid is less than about 70 percent, less than about 60 percent, or less than about 50 weight percent, on a dry weight basis. Example lipid weight ranges include about 10 to about 70 dry weight percent, such as about 20 to about 50 dry weight percent. In some embodiments, the amount of lipid is about 20, about 25, about 30, about 35, about 40, about 45, or about 50 percent by weight of the individual composition.

In one embodiment, the effervescent composition, the non-effervescent composition, or both are meltable. In one embodiment, the meltable composition comprises up to about 50 dry weight percent of the lipid. In some embodiments, the lipid is an oil selected from the group consisting of palm oil, palm kernel oil, soybean oil, cottonseed oil, and combinations thereof, wherein the oil may be hydrogenated, partially hydrogenated, or non-hydrogenated.

### Active Ingredient

The effervescent composition, the non-effervescent composition, or both compositions, in certain embodiments, comprise at least one active ingredient. As used herein, an “active ingredient” refers to one or more substances belonging to any of the following categories: API (active pharmaceutical substances), food additives, natural medicaments, and naturally occurring substances that can have an effect on humans. Example active ingredients include any ingredient known to impact one or more biological functions within the body, such as ingredients that furnish pharmacological activity or other direct effect in the diagnosis, cure, mitigation, treatment, or prevention of disease, or which affect the structure or any function of the body of humans (e.g., provide a stimulating action on the central nervous system, have an energizing effect, an antipyretic or analgesic action, or an otherwise useful effect on the body). In some embodiments, the active ingredient may be of the type generally referred to as dietary supplements, nutraceuticals, “phytochemicals” or “functional foods”. These types of additives are sometimes defined in the art as encompassing substances typically available from naturally-occurring sources (e.g., botanical materials) that provide one or more advantageous biological effects (e.g., health promotion, disease prevention, or other medicinal properties), but are not classified or regulated as drugs.

Non-limiting examples of active ingredients include those falling in the categories of botanical ingredients, stimulants, amino acids, nicotine components, and/or pharmaceutical, nutraceutical, and medicinal ingredients (e.g., vitamins, such as A, B3, B6, B12, and C, and/or terpenes, cannabimimetics, and cannabinoids, such as tetrahydrocannabinol (THC) and cannabidiol (CBD)). Each of these categories is further described herein below. The particular choice of active ingredients will vary depending upon the desired flavor, texture, and desired characteristics of the particular product.

The particular percentages of active ingredients present will vary depending upon the desired characteristics of the particular product. Typically, an active ingredient or combination thereof is present in a total concentration of at least about 0.001% by weight of the individual effervescent and/or non-effervescent composition, such as in a range from about 0.001% to about 20%. In some embodiments, the active ingredient or combination of active ingredients is present in a concentration from about 0.1% w/w to about 10% by weight, such as, e.g., from about from about 0.5% w/w to about 10%, from about 1% to about 10%, or from about 1% to about 5% by weight, based on the total weight of the effervescent and/or non-effervescent composition. In some embodiments, the active ingredient or combination of active ingredients is present in a concentration of from about 0.001%, about 0.01%, about 0.1%, or about 1%, up to about 20% by weight, such as, e.g., from about from about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight, based on the total weight of the individual effervescent and/or non-effervescent composition. Further suitable ranges for specific active ingredients are provided herein below.

In certain embodiments, the effervescent composition, the non-effervescent composition, or both the effervescent composition and the non-effervescent composition does not contain an active ingredient. For example, in certain embodiments, the effervescent composition, the non-effervescent composition, or both the effervescent composition and the non-effervescent composition comprises one or more flavorants in lieu of an active ingredient.

### Botanical

In some embodiments, the active ingredient comprises a botanical ingredient. As used herein, the term “botanical ingredient” or “botanical” refers to any plant material or fungal-derived material, including plant material in its natural form and plant material derived from natural plant materials, such as extracts or isolates from plant materials or treated plant materials (e.g., plant materials subjected to heat treatment, fermentation, bleaching, or other treatment processes capable of altering the physical and/or chemical nature of the material). For the purposes of the present disclosure, a “botanical” includes, but is not limited to, “herbal materials,” which refer to seed-producing plants that do not develop persistent woody tissue and are often valued for their medicinal or sensory characteristics (e.g., teas or tisanes). Reference to botanical material as “non-tobacco” is intended to exclude tobacco materials (i.e., does not include any Nicotiana species). In some embodiments, the botanical material is in an encapsulated form.

When present, a botanical is typically at a concentration of from about 0.01% w/w to about 10% by weight, such as, e.g., from about from about 0.01% w/w, about 0.05%, about 0.1%, or about 0.5%, to about 1%, about 2%, about 3%, about 4%, about 5%, about 6%, about 7%, about 8%, about 9%, or about 10%, about 11%, about 12%, about 13%, about 14%, or about 15% by weight, based on the total weight of the individual effervescent composition and/or non-effervescent composition.

The botanical materials useful in the present disclosure may comprise, without limitation, any of the compounds and sources set forth herein, including mixtures thereof. Certain botanical materials of this type are sometimes referred to as dietary supplements, nutraceuticals, “phytochemicals” or “functional foods.” Certain botanicals, as the plant material or an extract thereof, have found use in traditional herbal medicine, and are described further herein. Non-limiting examples of botanicals or botanical-derived materials include ashwagandha, Bacopa monniera, baobab, basil, Centella asiatica, Chai-hu, chamomile, cherry blossom, chlorophyll, cinnamon, citrus, cloves, cocoa, cordyceps, curcumin, damiana, Dorstenia arifolia, Dorstenia odorata, essential oils, eucalyptus, fennel, Galphimia glauca, ginger, Ginkgo biloba, ginseng (e.g., Panax ginseng), green tea, Griffonia simplicifolia, guarana, hemp, hops, jasmine, Kaempferia parviflora (Thai ginseng), kava, lavender, lemon balm, lemongrass, licorice, lutein, maca, matcha, Nardostachys chinensis, oil-based extract of Viola odorata, peppermint, quercetin, resveratrol, Rhizoma gastrodiae, Rhodiola, rooibos, rose essential oil, rosemary, Sceletium tortuosum, Schisandra, Skullcap, spearmint extract, Spikenard, terpenes, tisanes, turmeric, Turnera aphrodisiaca, valerian, white mulberry, and Yerba mate. In some embodiments, the active ingredient comprises lemon balm extract. In some embodiments, the active ingredient comprises ginseng.

### Stimulants

In some embodiments, the active ingredient comprises one or more stimulants. As used herein, the term “stimulant” refers to a material that increases activity of the central nervous system and/or the body, for example, enhancing focus, cognition, vigor, mood, alertness, and the like. Non-limiting examples of stimulants include caffeine, theacrine, theobromine, and theophylline. Theacrine (1,3,7,9-tetramethyluric acid) is a purine alkaloid which is structurally related to caffeine, and possesses stimulant, analgesic, and anti-inflammatory effects. Present stimulants may be natural, naturally derived, or wholly synthetic. For example, certain botanical materials (guarana, tea, coffee, cocoa, and the like) may possess a stimulant effect by virtue of the presence of e.g., caffeine or related alkaloids, and accordingly are “natural” stimulants. By “naturally derived” is meant the stimulant (e.g., caffeine, theacrine) is in a purified form, outside its natural (e.g., botanical) matrix. For example, caffeine can be obtained by extraction and purification from botanical sources (e.g., tea). By “wholly synthetic”, it is meant that the stimulant has been obtained by chemical synthesis. In some embodiments, the active ingredient comprises caffeine. In some embodiments, the active ingredient is caffeine. In some embodiments, the caffeine is present in an encapsulated form. On example of an encapsulated caffeine is Vitashure®, available from Balchem Corp., 52 Sunrise Park Road, New Hampton, NY, 10958.

When present, a stimulant or combination of stimulants (e.g., caffeine, theacrine, and combinations thereof) is typically at a concentration of from about 0.1% w/w to about 15% by weight, such as, e.g., from about from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the total weight of the individual effervescent and/or non-effervescent composition.

### Amino Acids

In some embodiments, the active ingredient comprises an amino acid. As used herein, the term “amino acid” refers to an organic compound that contains amine (—NH₂) and carboxyl (—COOH) or sulfonic acid (SO₃H) functional groups, along with a side chain (R group), which is specific to each amino acid. Amino acids may be proteinogenic or non-proteinogenic. By “proteinogenic” is meant that the amino acid is one of the twenty naturally occurring amino acids found in proteins. The proteinogenic amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. By “non-proteinogenic” is meant that either the amino acid is not found naturally in protein, or is not directly produced by cellular machinery (e.g., is the product of post-tranlational modification). Non-limiting examples of non-proteinogenic amino acids include gamma-aminobutyric acid (GABA), taurine (2-aminoethanesulfonic acid), theanine (L-γ-glutamylethylamide), hydroxyproline, and beta-alanine. In some embodiments, the active ingredient comprises an amino acid selected from one or more of arginine, beta-alanine, carnitine, choline, creatine, GABA, glutamic acid, lysine, magnesium threonate, phenylalanine, tryptophan, tyrosine, and combination thereof. In some embodiments, the active ingredient comprises theanine. In some embodiments, the e active ingredient comprises GABA. In some embodiments, the active ingredient comprises taurine.

When present, an amino acid or combination of amino acids (e.g., taurine, theanine, GABA, or combinations thereof) is typically at a concentration of from about 0.1% w/w to about 15% by weight, such as, e.g., from about from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the total weight of the individual effervescent and/or non-effervescent composition.

### Vitamins and Minerals

In some embodiments, the active ingredient comprises a vitamin or combination of vitamins. As used herein, the term “vitamin” refers to an organic molecule (or related set of molecules) that is an essential micronutrient needed for the proper functioning of metabolism in a mammal. There are thirteen vitamins required by human metabolism, which are: vitamin A (as all-trans-retinol, all-trans-retinyl-esters, as well as all-trans-beta-carotene and other provitamin A carotenoids), vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folic acid or folate), vitamin B12 (cobalamins), vitamin C (ascorbic acid), vitamin D (calciferols), vitamin E (tocopherols and tocotrienols), and vitamin K (quinones). In some embodiments, the active ingredient comprises vitamin C.

When present, a vitamin or combination of vitamins (e.g., vitamin B6, vitamin B12, vitamin E, vitamin C, or a combination thereof) is typically at a concentration of from about 0.01% w/w to about 10% by weight, such as, e.g., from about from about 0.01%, about 0.02%, about 0.03%, about 0.04%, about 0.05%, about 0.06%, about 0.07%, about 0.08%, about 0.09%, about 0.1% w/w, about 0.2%, about 0.3%, about 0.4%, about 0.5% about 0.6%, about 0.7%, about 0.8%, about 0.9%, or about 1%, to about 2%, about 3%, about 4%, about 5%, about 6%, about 7%, about 8%, about 9%, or about 10% by weight, based on the total weight of the individual effervescent and/or non-effervescent composition.

In some embodiments, the active ingredient comprises a mineral. As used herein, the term “mineral” refers to an inorganic molecule (or related set of molecules) that is an essential micronutrient needed for the proper functioning of various systems in a mammal. Non-limiting examples of minerals include iron, zinc, copper, selenium, chromium, cobalt, manganese, calcium, phosphorus, sulfur, magnesium, and the like. In some embodiments, the active ingredient comprises iron. Suitable sources of iron include, but are not limited to, ferrous salts such as ferrous sulfate and ferrous gluconate. In some embodiments, the iron is encapsulated.

### Nicotine Component

In certain embodiments, the active ingredient comprises a nicotine component. By “nicotine component” is meant any suitable form of nicotine (e.g., free base or salt, natural or synthetic) for providing oral absorption of at least a portion of the nicotine present. Typically, the nicotine component is selected from the group consisting of nicotine free base and a nicotine salt. In some embodiments, the nicotine component is nicotine in its free base form, which easily can be adsorbed in for example, a microcrystalline cellulose material to form a microcrystalline cellulose-nicotine carrier complex. See, for example, the discussion of nicotine in free base form in U.S. Pat. Pub. No. 2004/0191322 to Hansson, which is incorporated herein by reference.

In some embodiments, at least a portion of the nicotine component can be employed in the form of a salt. Salts of nicotine can be provided using the types of ingredients and techniques set forth in U.S. Pat. No. 2,033,909 to Cox et al. and Perfetti, Beitrage Tabakforschung Int., 12: 43-54 (1983), which are incorporated herein by reference. Additionally, salts of nicotine are available from sources such as Pfaltz and Bauer, Inc. and K&K Laboratories, Division of ICN Biochemicals, Inc. Typically, the nicotine component is selected from the group consisting of nicotine free base, a nicotine salt such as hydrochloride, dihydrochloride, monotartrate, bitartrate, sulfate, salicylate, and nicotine zinc chloride.

In some embodiments, at least a portion of the nicotine can be in the form of a resin complex of nicotine, where nicotine is bound in an ion-exchange resin, such as nicotine polacrilex, which is nicotine bound to, for example, a polymethacrilic acid, such as Amberlite IRP64, Purolite C₁₁₅HMR, or Doshion P551. See, for example, U.S. Pat. No. 3,901,248 to Lichtneckert et al., which is incorporated herein by reference. Another example is a nicotine-polyacrylic carbomer complex, such as with Carbopol 974P. In some embodiments, nicotine may be present in the form of a nicotine polyacrylic complex.

Typically, the nicotine component (calculated as the free base) when present, is in a concentration of at least about 0.001% by weight of the effervescent or non-effervescent composition, such as in a range from about 0.001% to about 10%. In some embodiments, the nicotine component is present in a concentration from about 0.1% w/w to about 10% by weight, such as, e.g., from about from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, or about 10% by weight, calculated as the free base and based on the total weight of the effervescent composition or non-effervescent. In some embodiments, the nicotine component is present in a concentration from about 0.1% w/w to about 3% by weight, such as, e.g., from about from about 0.1% w/w to about 2.5%, from about 0.1% to about 2.0%, from about 0.1% to about 1.5%, or from about 0.1% to about 1% by weight, calculated as the free base and based on the total weight of the individual effervescent or non-effervescent composition.

In some embodiments, the products or compositions of the disclosure can be characterized as completely free or substantially free of any nicotine component (e.g., any embodiment as disclosed herein may be completely or substantially free of any nicotine component). By “substantially free” is meant that no nicotine has been intentionally added, beyond trace amounts that may be naturally present in e.g., a botanical material. For example, certain embodiments can be characterized as having less than 0.001% by weight of nicotine, or less than 0.0001%, or even 0% by weight of nicotine, calculated as the free base.

In some embodiments, the active ingredient comprises a nicotine component (e.g., any effervescent and/or non-effervescent composition of the disclosure, in addition to comprising any active ingredient or combination of active ingredients as disclosed herein, may further comprise a nicotine component). In certain embodiments, the effervescent composition and the non-effervescent composition both comprise a nicotine component.

### Cannabinoids

In some embodiments, the active ingredient comprises one or more cannabinoids. As used herein, the term “cannabinoid” refers to a class of diverse chemical compounds that acts on cannabinoid receptors, also known as the endocannabinoid system, in cells that alter neurotransmitter release in the brain. Ligands for these receptor proteins include the endocannabinoids produced naturally in the body by animals; phytocannabinoids, found in cannabis; and synthetic cannabinoids, manufactured artificially. Cannabinoids found in cannabis include, without limitation: cannabigerol (CBG), cannabichromene (CBC), cannabidiol (CBD), tetrahydrocannabinol (THC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), and tetrahydrocannabivarinic acid (THCV A). In certain embodiments, the cannabinoid is selected from tetrahydrocannabinol (THC), the primary psychoactive compound in cannabis, and cannabidiol (CBD) another major constituent of the plant, but which is devoid of psychoactivity. All of the above compounds can be used in the form of an isolate from plant material or synthetically derived.

In some embodiments, the cannabinoid is selected from the group consisting of cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), tetrahydrocannabivarinic acid (THCV A), and mixtures thereof. In some embodiments, the cannabinoid comprises at least tetrahydrocannabinol (THC). In some embodiments, the cannabinoid is tetrahydrocannabinol (THC). In some embodiments, the cannabinoid comprises at least cannabidiol (CBD). In some embodiments, the cannabinoid is cannabidiol (CBD). In some embodiments, the CBD is synthetic CBD. The choice of cannabinoid and the particular percentages thereof which may be present within the disclosed compositions will vary depending upon the desired flavor, texture, and other characteristics of the overall product.

Alternatively, the active ingredient can be a cannabimimetic, which is a class of compounds derived from plants other than cannabis that have biological effects on the endocannabinoid system similar to cannabinoids. Examples include yangonin, alpha-amyrin or beta-amyrin (also classified as terpenes), cyanidin, curcumin (tumeric), catechin, quercetin, salvinorin A, N-acylethanolamines, and N-alkylamide lipids. Such compounds can be used in the same amounts and ratios noted herein for cannabinoids.

When present, a cannabinoid (e.g., CBD) or cannabimimetic is typically in a concentration of at least about 0.1% by weight of the individual effervescent and/or non-effervescent composition, such as in a range from about 0.1% to about 30%, such as, e.g., from about from about 0.1%, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 15%, about 20%, or about 30% by weight, based on the total weight of the individual composition. In some embodiments, the effervescent and/or non-effervescent composition as disclosed herein comprises CBD in an amount from about 0.1 to about 30% by weight, or from about 1 to about 20% by weight, based on the total weight of the individual effervescent and/or non-effervescent composition.

### Terpenes

Active ingredients suitable for use in the present disclosure can also be classified as terpenes, many of which are associated with biological effects, such as calming effects. Terpenes are understood to have the general formula of (C₅H₈)ₙ and include monoterpenes, sesquiterpenes, and diterpenes. Terpenes can be acyclic, monocyclic or bicyclic in structure. Some terpenes provide an entourage effect when used in combination with cannabinoids or cannabimimetics. Examples include beta-caryophyllene, linalool, limonene, beta-citronellol, linalyl acetate, pinene (alpha or beta), geraniol, carvone, eucalyptol, menthone, iso-menthone, piperitone, myrcene, beta-bourbonene, and germacrene, which may be used singly or in combination.

In some embodiments, the terpene is a terpene derivable from a phytocannabinoid producing plant, such as a plant from the stain of the Cannabis sativa species, such as hemp. Suitable terpenes in this regard include so-called “C₁₀” terpenes, which are those terpenes comprising 10 carbon atoms, and so-called “C₁₅” terpenes, which are those terpenes comprising 15 carbon atoms. In some embodiments, the active ingredient comprises more than one terpene. For example, the active ingredient may comprise one, two, three, four, five, six, seven, eight, nine, ten or more terpenes as defined herein. In some embodiments, the terpene is selected from pinene (alpha and beta), geraniol, linalool, limonene, carvone, eucalyptol, menthone, iso-menthone, piperitone, myrcene, beta-bourbonene, germacrene and mixtures thereof.

### Pharmaceutical Ingredients

In some embodiments, the active ingredient comprises a pharmaceutical ingredient. The pharmaceutical ingredient can be any known agent adapted for therapeutic, prophylactic, or diagnostic use. These can include, for example, synthetic organic compounds, proteins and peptides, polysaccharides and other sugars, lipids, phospholipids, inorganic compounds (e.g., magnesium, selenium, zinc, nitrate), neurotransmitters or precursors thereof (e.g., serotonin, 5-hydroxy-tryptophan, oxitriptan, acetylcholine, dopamine, melatonin), and nucleic acid sequences, having therapeutic, prophylactic, or diagnostic activity. Non-limiting examples of pharmaceutical ingredients include analgesics and antipyretics (e.g., acetylsalicylic acid, acetaminophen, 3-(4-isobutylphenyl)propanoic acid), phosphatidylserine, myoinositol, docosahexaenoic acid (DHA, Omega-3), arachidonic acid (AA, Omega-6), S-adenosylmethionine (SAM), beta-hydroxy-beta-methylbutyrate (HMB), citicoline (cytidine-5′-diphosphate-choline), and cotinine. In some embodiments, the active ingredient comprises citicoline. In some embodiments, the active ingredient comprises sunflower lecithin.

The amount of pharmaceutical ingredient may vary. For example, when present, a pharmaceutical ingredient is typically at a concentration of from about 0.001% w/w to about 10% by weight, such as, e.g., from about from about 0.01%, about 0.02%, about 0.03%, about 0.04%, about 0.05%, about 0.06%, about 0.07%, about 0.08%, about 0.09%, about 0.1% w/w, about 0.2%, about 0.3%, about 0.4%, about 0.5% about 0.6%, about 0.7%, about 0.8%, about 0.9%, or about 1%, to about 2%, about 3%, about 4%, about 5%, about 6%, about 7%, about 8%, about 9%, or about 10% by weight, based on the total weight of the individual effervescent and/or non-effervescent composition.

In some embodiments, the active ingredient as described herein may be sensitive to degradation (e.g., oxidative, photolytic, thermal, evaporative) during processing or upon storage of the oral product. In such embodiments, the active ingredient (such as caffeine, vitamin A, and iron (Fe)) may be encapsulated, or the matrix otherwise modified with fillers, binders, and the like, to provide enhanced stability to the active ingredient. For example, binders such as functional celluloses (e.g., cellulose ethers including, but not limited to, hydroxypropyl cellulose) may be employed to enhance stability of such actives toward degradation. Additionally, encapsulated actives may need to be paired with an excipient in the composition to increase their solubility and/or bioavailability. Non-limiting examples of suitable excipients include beta-carotene, lycopene, Vitamin D, Vitamin E, Co-enzyme Q 10, Vitamin K, and curcumin.

In other embodiments, in order to provide a desired concentration of the active ingredient by weight, an initial quantity of the active ingredient may be increased to compensate for a gradual degradative loss. Accordingly, larger initial amounts than those disclosed herein are contemplated by the present disclosure.

### Ion Pairing of Active Ingredient and Organic Acid

In some embodiments, the active ingredient comprises a basic amine (e.g., nicotine). In some such embodiments, the composition comprising the active ingredient comprising a basic amine may further comprise an organic acid, and the basic amine and the organic acid may at least in part be associated in the form of an ion pair and/or salt between the basic amine and the organic acid or a conjugate base of the organic acid. Accordingly, in some embodiments, the effervescent composition and/or the non-effervescent composition comprise a basic amine and an organic acid, an alkali metal salt of an organic acid, or a combination thereof, and at least a portion of the basic amine is associated with at least a portion of the organic acid or the alkali metal salt thereof, the association in the form of a basic amine-organic acid salt, an ion pair between the basic amine and a conjugate base of the organic acid, or both. In some embodiments, at least a portion of the basic amine is associated with at least a portion of the organic acid or the alkali metal salt thereof. Depending on multiple variables (concentration, pH, nature of the organic acid, and the like), the basic amine present in the composition can exist in multiple forms, including ion paired, in solution (i.e., fully solvated), as the free base, as a cation, as a salt, or any combination thereof. In some embodiments, the association between the basic amine and at least a portion of the organic acid or the alkali metal salt thereof is in the form of an ion pair between the basic amine and a conjugate base of the organic acid.

Ion pairing describes the partial association of oppositely charged ions in relatively concentrated solutions to form distinct chemical species called ion pairs. The strength of the association (i.e., the ion pairing) depends on the electrostatic force of attraction between the positive and negative ions (i.e., protonated basic amine and the conjugate base of the organic acid). By “conjugate base” is meant the base resulting from deprotonation of the corresponding acid (e.g., benzoate is the conjugate base of benzoic acid). On average, a certain population of these ion pairs exists at any given time, although the formation and dissociation of ion pairs is continuous. In the composition as disclosed herein, and/or upon oral use of said composition (e.g., upon contact with saliva), the basic amine and the conjugate base of the organic acid exist at least partially in the form of an ion pair. Without wishing to be bound by theory, it is believed that such ion pairing may minimize chemical degradation of the basic amine and/or enhance the oral availability of the basic amine. At alkaline pH values (e.g., such as from about 7.5 to about 9), certain basic amines, for example nicotine, are largely present in the free base form, which has relatively low water solubility, and low stability with respect to evaporation and oxidative decomposition, but high mucosal availability. Conversely, at acidic pH values (such as from about 6.5 to about 4), certain basic amines, for example nicotine, are largely present in a protonated form, which has relatively high water solubility, and higher stability with respect to evaporation and oxidative decomposition, but low mucosal availability. Surprisingly, according to the present disclosure, it has been found that the properties of stability, solubility, and availability of the nicotine in a composition configured for oral use can be mutually enhanced through ion pairing or salt formation of nicotine with appropriate organic acids and/or their conjugate bases. Specifically, nicotine-organic acid ion pairs of moderate lipophilicity result in favorable stability and absorption properties. Lipophilicity is conveniently measured in terms of logP, the partition coefficient of a molecule between a lipophilic phase and an aqueous phase, usually octanol and water, respectively. An octanol-water partitioning favoring distribution of a basic amine-organic acid ion pair into octanol is predictive of good absorption of the basic amine present in the composition through the oral mucosa.

### Basic Amine

By “basic amine” is meant a molecule including at least one basic amine functional group. Examples of basic amines include, but are not limited to, alkaloids. By “basic amine functional group” is meant a group containing a nitrogen atom having a lone pair of electrons. The basic amine functional group is attached to or incorporated within the molecule through one or more covalent bonds to the said nitrogen atom. The basic amine may be a primary, secondary, or tertiary amine, meaning the nitrogen bears one, two, or three covalent bonds to carbon atoms. By virtue of the lone pair of electrons on the nitrogen atom, such amines are termed “basic”, meaning the lone electron pair is available for hydrogen bonding. The basicity (i.e., the electron density on the nitrogen atom and consequently the availability and strength of hydrogen bonding to the nitrogen atom) of the basic amine may be influenced by the nature of neighboring atoms, the steric bulk of the molecule, and the like. Generally, the basic amine is released from the composition and absorbed through the oral mucosa, thereby entering the blood stream, where it is circulated systemically. Generally, the basic amine is present in or as an active ingredient in the composition, as described herein. In some embodiments, the basic amine is caffeine. In some embodiments, the basic amine is nicotine or a nicotine component. Typically, the nicotine component is selected from the group consisting of nicotine free base, nicotine as an ion pair, and a nicotine salt. In some embodiments, at least a portion of the nicotine is in its free base form. In some embodiments, at least a portion of the nicotine is present as a nicotine salt, or at least a portion of the nicotine is present as an ion pair with at least a portion of the organic acid or the conjugate base thereof, as disclosed herein above. More information regarding a nicotine component is set forth below.

### Organic Acid

As used herein, the term “organic acid” refers to an organic (i.e., carbon-based) compound that is characterized by acidic properties. Typically, organic acids are relatively weak acids (i.e., they do not dissociate completely in the presence of water), such as carboxylic acids (—CO₂H) or sulfonic acids (—SO₂OH). As used herein, reference to organic acid means an organic acid that is intentionally added. In this regard, an organic acid may be intentionally added as a specific composition ingredient as opposed to merely being inherently present as a component of another composition ingredient (e.g., the small amount of organic acid which may inherently be present in a composition ingredient, such as a tobacco material).

Suitable organic acids will typically have a range of lipophilicities (i.e., a polarity giving an appropriate balance of water and organic solubility). Typically, lipophilicities of suitable organic acids, as indicated by logP, will vary between about 1 and about 12 (more soluble in octanol than in water). In some embodiments, the organic acid has a logP value of from about 3 to about 12, e.g., from about 3.0, about 3.5, about 4.0, about 4.5, about 5.0, about 5.5, about 6.0, about 6.5, about 7.0, about 7.5, or about 8.0, to about 8.5, about 9.0, about 9.5, about 10.0, about 10.5, about 11.0, about 11.5, or about 12.0. In certain embodiments, lipophilicities of suitable organic acids, as indicated by logP, will vary between about 1.4 and about 4.5 (more soluble in octanol than in water). In some embodiments, the organic acid has a logP value of from about 1.5 to about 4.0, e.g., from about 1.5, about 2.0, about 2.5, or about 3.0, to about 3.5, about 4.0, about 4.5, or about 5.0. Particularly suitable organic acids have a logP value of from about 1.7 to about 4, such as from about 2.0, about 2.5, or about 3.0, to about 3.5, or about 4.0. In specific embodiments, the organic acid has a logP value of about 2.5 to about 3.5. In some embodiments, organic acids outside this range may also be utilized for various purposes and in various amounts, as described further herein below. For example, in some embodiments, the organic acid may have a logP value of greater than about 4.5, such as from about 4.5 to about 12.0. Particularly, the presence of certain solvents or solubilizing agents (e.g., inclusion in the composition of glycerin or propylene glycol) may extend the range of lipophilicity (i.e., values of logP higher than 4.5, such as from about 4.5 to about 12.0).

Without wishing to be bound by theory, it is believed that moderately lipophilic organic acids (e.g., logP of from about 1.4 to about 4.5) produce ion pairs with nicotine which are of a polarity providing good octanol-water partitioning of the ion pair, and hence partitioning of nicotine, into octanol versus water. As discussed above, such partitioning into octanol is predictive of favorable oral availability.

In some embodiments, the organic acid is a carboxylic acid or a sulfonic acid. The carboxylic acid or sulfonic acid functional group may be attached to any alkyl, cycloalkyl, heterocycloalkyl, aryl, or heteroaryl group having, for example, from one to twenty carbon atoms (C₁-C₂₀). In some embodiments, the organic acid is an alkyl, cycloalkyl, heterocycloalkyl, aryl, or heteroaryl carboxylic or sulfonic acid.

As used herein, “alkyl” refers to any straight chain or branched chain hydrocarbon. The alkyl group may be saturated (i.e., having all sp³ carbon atoms), or may be unsaturated (i.e., having at least one site of unsaturation). As used herein, the term “unsaturated” refers to the presence of a carbon-carbon, sp² double bond in one or more positions within the alkyl group. Unsaturated alkyl groups may be mono- or polyunsaturated. Representative straight chain alkyl groups include, but are not limited to, methyl, ethyl, n-propyl, n-butyl, n-pentyl, and n-hexyl. Branched chain alkyl groups include, but are not limited to, isopropyl, sec-butyl, isobutyl, tert-butyl, isopentyl, and 2-methylbutyl. Representative unsaturated alkyl groups include, but are not limited to, ethylene or vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like. An alkyl group can be unsubstituted or substituted.

“Cycloalkyl” as used herein refers to a carbocyclic group, which may be mono- or bicyclic. Cycloalkyl groups include rings having 3 to 7 carbon atoms as a monocycle or 7 to 12 carbon atoms as a bicycle. Examples of monocyclic cycloalkyl groups include cyclopropyl, cyclobutyl, cyclopentyl, cyclohexyl, cycloheptyl, and cyclooctyl. A cycloalkyl group can be unsubstituted or substituted, and may include one or more sites of unsaturation (e.g., cyclopentenyl or cyclohexenyl).

The term “aryl” as used herein refers to a carbocyclic aromatic group. Examples of aryl groups include, but are not limited to, phenyl and naphthyl. An aryl group can be unsubstituted or substituted.

“Heteroaryl” and “heterocycloalkyl” as used herein refer to an aromatic or non-aromatic ring system, respectively, in which one or more ring atoms is a heteroatom, e.g. nitrogen, oxygen, and sulfur. The heteroaryl or heterocycloalkyl group comprises up to 20 carbon atoms and from 1 to 3 heteroatoms selected from N, O, and S. A heteroaryl or heterocycloalkyl may be a monocycle having 3 to 7 ring members (for example, 2 to 6 carbon atoms and 1 to 3 heteroatoms selected from N, O, and S) or a bicycle having 7 to 10 ring members (for example, 4 to 9 carbon atoms and 1 to 3 heteroatoms selected from N, O, and S), for example: a bicyclo[4,5], [5,5], [5,6], or [6,6] system. Examples of heteroaryl groups include by way of example and not limitation, pyridyl, thiazolyl, tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, benzotriazolyl, benzisoxazolyl, and isatinoyl. Examples of heterocycloalkyls include by way of example and not limitation, dihydroypyridyl, tetrahydropyridyl (piperidyl), tetrahydrothiophenyl, piperidinyl, 4-piperidonyl, pyrrolidinyl, 2-pyrrolidonyl, tetrahydrofuranyl, tetrahydropyranyl, bis-tetrahydropyranyl, tetrahydroquinolinyl, tetrahydroisoquinolinyl, decahydroquinolinyl, octahydroisoquinolinyl, piperazinyl, quinuclidinyl, and morpholinyl. Heteroaryl and heterocycloalkyl groups can be unsubstituted or substituted.

“Substituted” as used herein and as applied to any of the above alkyl, aryl, cycloalkyl, heteroaryl, heterocyclyl, means that one or more hydrogen atoms are each independently replaced with a substituent. Typical substituents include, but are not limited to, —Cl, Br, F, alkyl, —OH, —OCH₃, NH₂, —NHCH₃, —N(CH₃)₂, —CN, —NC(═O)CH₃, —C(═O)—, —C(═O)NH₂, and —C(═O)N(CH₃)₂. Wherever a group is described as “optionally substituted,” that group can be substituted with one or more of the above substituents, independently selected for each occasion. In some embodiments, the substituent may be one or more methyl groups or one or more hydroxyl groups.

In some embodiments, the organic acid is an alkyl carboxylic acid. Non-limiting examples of alkyl carboxylic acids include formic acid, acetic acid, propionic acid, butyric acid, valeric acid, caproic acid, heptanoic acid, octanoic acid, nonanoic acid, decanoic acid, undecanoic acid, dodecanoic acid, stearic acid, oleic acid, linoleic acid, linolenic acid, and the like.

In some embodiments, the organic acid is an alkyl sulfonic acid. Non-limiting examples of alkyl sulfonic acids include propanesulfonic acid, heptanesulfonic acid, and octanesulfonic acid.

In some embodiments, the alkyl carboxylic or sulfonic acid is substituted with one or more hydroxyl groups. Non-limiting examples include glycolic acid, 4-hydroxybutyric acid, and lactic acid.

In some embodiments, an organic acid may include more than one carboxylic acid group or more than one sulfonic acid group (e.g., two, three, or more carboxylic acid groups). Non-limiting examples include oxalic acid, fumaric acid, maleic acid, and glutaric acid. In organic acids containing multiple carboxylic acids (e.g., from two to four carboxylic acid groups), one or more of the carboxylic acid groups may be esterified. Non-limiting examples include succinic acid monoethyl ester, monomethyl fumarate, monomethyl or dimethyl citrate, and the like.

In some embodiments, the organic acid may include more than one carboxylic acid group and one or more hydroxyl groups. Non-limiting examples of such acids include tartaric acid, citric acid, and the like.

In some embodiments, the organic acid is an aryl carboxylic acid or an aryl sulfonic acid. Non-limiting examples of aryl carboxylic and sulfonic acids include benzoic acid, toluic acids, salicylic acid, benzenesulfonic acid, and p-toluenesulfonic acid.

Further non-limiting examples of organic acids which may be useful in certain embodiments include 2,2-dichloroacetic acid, 2-hydroxyethanesulfonic acid, 2-oxoglutaric acid, 4-acetamidobenzoic acid, 4-aminosalicylic acid, adipic acid, ascorbic acid (L), aspartic acid (L), alpha-methylbutyric acid, camphoric acid (+), camphor-10-sulfonic acid (+), cinnamic acid, cyclamic acid, dodecylsulfuric acid, ethane-1,2-disulfonic acid, ethanesulfonic acid, furoic acid, galactaric acid, gentisic acid, glucoheptonic acid, gluconic acid, glucuronic acid, glutamic acid, glycerophosphoric acid, glycolic acid, hippuric acid, isobutyric acid, isovaleric acid, lactobionic acid, lauric acid, levulinic acid, malic acid, malonic acid, mandelic acid, methanesulfonic acid, naphthalene-1,5-disulfonic acid, naphthalene-2-sulfonic acid, oleic acid, palmitic acid, pamoic acid, phenylacetic acid, pyroglutamic acid, pyruvic acid, sebacic acid, stearic acid, and undecylenic acid.

Examples of suitable acids include, but are not limited to, the list of organic acids in Table 1.

TABLE 1
Non-limiting examples of suitable organic acids

| Acid Name | log(P) |
|---|---|
| benzoic acid | 1.9 |
| phenylacetic | 1.4 |
| p-toluic acid | 2.3 |
| ethyl benzoic acid | 2.9 |
| isopropyl benzoic acid | 3.5 |
| 4-phenylbutyric | 2.4 |
| 2-napthoxyacetic acid | 2.5 |
| napthylacetic acid | 2.7 |
| heptanoic acid | 2.5 |
| octanoic acid | 3.05 |
| nonanoic acid | 3.5 |
| decanoic acid | 4.09 |
| 9-deceneoic acid | 3.3 |
| 2-deceneoic acid | 3.8 |
| 10-undecenoic acid | 3.9 |
| dodecandioic acid | 3.2 |
| dodecanoic acid | 4.6 |
| myristic acid | 5.3 |
| palmitic acid | 6.4 |
| stearic acid | 7.6 |
| cyclohexanebutanoic acid | 3.4 |
| 1-heptanesulfonic acid | 2.0 |
| 1-octanesulfonic acid | 2.5 |
| 1-nonanesulfonic acid | 3.1 |
| monooctyl succinate | 2.8 |
| tocopherol succinate | 10.2 |
| monomenthyl succinate | 3 |
| monomenthyl glutarate | 3.4 |
| norbixin | 7.2 |
| ((2E,4E,6E,8E,10E,12E,14E,16E,18E)- | |
| 4,8,13,17-tetramethylicosa- | |
| 2,4,6,8,10,12,14,16,18-nonaenedioic | |
| acid) | |
| bixin | 7.5 |
| ((2E,4E,6E,8E,10E,12E,14E,16Z,18E)- | |
| 20-methoxy-4,8,13,17-tetramethyl-20- | |
| oxoicosa-2,4,6,8,10,12,14,16,18- | |
| nonaenoic acid) | |

In some embodiments, the organic acid is benzoic acid, a toluic acid, benzenesulfonic acid, toluenesulfonic acid, hexanoic acid, heptanoic acid, decanoic acid, or octanoic acid. In some embodiments, the organic acid is benzoic acid, octanoic acid, or decanoic acid. In some embodiments, the organic acid is octanoic acid.

In some embodiments, the organic acid is a mono ester of a di- or poly-acid, such as mono-octyl succinate, mono-octyl fumarate, or the like. For example, the organic acid is a mono ester of a dicarboxylic acid or a poly-carboxylic acid. In some embodiments, the dicarboxylic acid is malonic acid, succinic acid, glutaric acid, adipic acid, fumaric acid, maleic acid, or a combination thereof. In some embodiments, the dicarboxylic acid is succinic acid, glutaric acid, fumaric acid, maleic acid, or a combination thereof. In some embodiments, the dicarboxylic acid is succinic acid, glutaric acid, or a combination thereof.

In some embodiments, the alcohol forming the mono ester of the dicarboxylic acid is a lipophilic alcohol. Examples of suitable lipophilic alcohols include, but are not limited to, octanol, menthol, and tocopherol. In some embodiments, the organic acid is an octyl mono ester of a dicarboxylic acid, such as monooctyl succinate, monooctyl fumarate, or the like. In some embodiments, the organic acid is a monomenthyl ester of a dicarboxylic acid. Certain menthyl esters may be desirable in oral compositions as described herein by virtue of the cooling sensation they may provide upon use of the product comprising the composition. In some embodiments, the organic acid is monomenthyl succinate, monomenthyl fumarate, monomenthyl glutarate, or a combination thereof. In some embodiments, the organic acid is a monotocopheryl ester of a dicarboxylic acid. Certain tocopheryl esters may be desirable in oral compositions as described herein by virtue of the antioxidant effects they may provide. In some embodiments, the organic acid is tocopheryl succinate, tocopheryl fumarate, tocopheryl glutarate, or a combination thereof.

In some embodiments, the organic acid is a carotenoid derivative having one or more carboxylic acids. Carotenoids are tetraterpenes, meaning that they are produced from 8 isoprene molecules and contain 40 carbon atoms. Accordingly, they are usually lipophilic due to the presence of long unsaturated aliphatic chains, and are generally yellow, orange, or red in color. Certain carotenoid derivatives can be advantageous in oral compositions by virtue of providing both ion pairing and serving as a colorant in the composition. In some embodiments, the organic acid is 2E,4E,6E,8E,10E,12E,14E,16Z,18E)-20-methoxy-4,8,13,17-tetramethyl-20-oxoicosa-2,4,6,8,10,12,14,16,18-nonaenoic acid (bixin) or an isomer thereof. Bixin is an apocarotenoid found in annatto seeds from the achiote tree (Bixa orellana), and is the naturally occurring pigment providing the reddish orange color to annatto. Bixin is soluble in fats and alcohols but insoluble in water, and is chemically unstable when isolated, converting via isomerization into the double bond isomer, trans-bixin (β-bixin), having the structure:

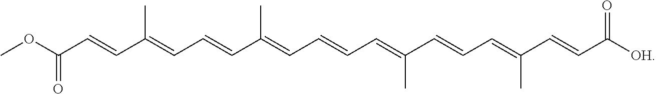 [SMILES: COC(=O)/C=C/C(C)=C/C=C/C(C)=C/C=C/C=C(C)/C=C/C=C(C)/C=C/C(=O)O]

In some embodiments, the organic acid is (2E,4E,6E,8E,10E,12E,14E,16E,18E)-4,8,13,17-tetramethylicosa-2,4,6,8,10,12,14,16,18-nonaenedioic acid (norbixin), a water soluble hydrolysis product of bixin having the structure:

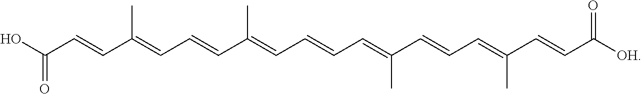 [SMILES: CC(/C=C/C=C(C)/C=C/C(=O)O)=C\C=C\C=C(C)\C=C\C=C(C)\C=C\C(=O)O]

The selection of organic acid may further depend on additional properties in addition to or without consideration to the logP value. For example, an organic acid should be one recognized as safe for human consumption, and which has acceptable flavor, odor, volatility, stability, and the like. Determination of appropriate organic acids is within the purview of one of skill in the art.

In some embodiments, more than one organic acid may be present. For example, the composition may comprise two, or three, or four, or more organic acids. Accordingly, reference herein to “an organic acid” contemplates mixtures of two or more organic acids. The relative amounts of the multiple organic acids may vary. For example, a composition may comprise equal amounts of two, or three, or more organic acids, or may comprise different relative amounts. In this manner, it is possible to include certain organic acids (e.g., citric acid or myristic acid) which have a logP value outside the desired range, when combined with other organic acids to provide the desired average logP range for the combination. In some embodiments, it may be desirable to include organic acids in the composition which have logP values outside the desired range for purposes such as, but not limited to, providing desirable organoleptic properties, stability, as flavor components, and the like. Further, certain lipophilic organic acids have undesirable flavor and or aroma characteristics which would preclude their presence as the sole organic acid (e.g., in equimolar or greater quantities relative to nicotine). Without wishing to be bound by theory, it is believed that a combination of different organic acids may provide the desired ion pairing while the concentration of any single organic acid in the composition remains below the threshold which would be found objectionable from a sensory perspective.

For example, in some embodiments, the organic acid may comprise from about 1 to about 5 or more molar equivalents of benzoic acid relative to nicotine, combined with e.g., about 0.2 molar equivalents of octanoic acid or a salt thereof, and 0.2 molar equivalents of decanoic acid or a salt thereof.

In some embodiments, the organic acid is a combination of any two organic acids selected from the group consisting of benzoic acid, a toluic acid, benzenesulfonic acid, toluenesulfonic acid, hexanoic acid, heptanoic acid, decanoic acid, and octanoic acid. In some embodiments, the organic acid is a combination of benzoic acid, octanoic acid, and decanoic acid, or benzoic and octanoic acid. In some embodiments, the composition comprises citric acid in addition to one or more of benzoic acid, a toluic acid, benzenesulfonic acid, toluenesulfonic acid, hexanoic acid, heptanoic acid, decanoic acid, and octanoic acid.

In some embodiments, the composition comprises an alkali metal salt of an organic acid. For example, at least a portion of the organic acid may be present in the composition in the form of an alkali metal salt. Suitable alkali metal salts include lithium, sodium, and potassium. In some embodiments, the alkali metal is sodium or potassium. In some embodiments, the alkali metal is sodium. In some embodiments, the composition comprises an organic acid and a sodium salt of the organic acid.

In some embodiments, the composition comprises benzoic acid and sodium benzoate, octanoic acid and sodium octanoate, decanoic acid and sodium decanoate, or a combination thereof.

In some embodiments, the ratio of the organic acid to the sodium salt (or other alkali metal) of the organic acid is from about 0.1 to about 10, such as from about 0.1, about 0.25, about 0.3, about 0.5, about 0.75, or about 1, to about 2, about 5, or about 10. For example, in some embodiments, both an organic acid and the sodium salt thereof are added to the other components of the composition, wherein the organic acid is added in excess of the sodium salt, in equimolar quantities with the sodium salt, or as a fraction of the sodium salt. One of skill in the art will recognize that the relative amounts will be determined by the desired pH of the composition, as well as the desired ionic strength. For example, the organic acid may be added in a quantity to provide a desired pH level of the composition, while the alkali metal (e.g., sodium) salt is added in a quantity to provide the desired extent of ion pairing. As one of skill in the art will understand, the quantity of organic acid (i.e., the protonated form) present in the composition, relative to the alkali metal salt or conjugate base form present in the composition, will vary according to the pH of the composition and the pKa of the organic acid, as well as according to the actual relative quantities initially added to the composition.

The amount of organic acid or an alkali metal salt thereof present in the composition, relative to nicotine, may vary. Generally, as the concentration of the organic acid (or the conjugate base thereof) increases, the percent of nicotine that is ion paired with the organic acid increases. This typically increases the partitioning of the nicotine, in the form of an ion pair, into octanol versus water as measured by the logP (the login of the partitioning coefficient). In some embodiments, the composition comprises from about 0.05, about 0.1, about 1, about 1.5, about 2, or about 5, to about 10, about 15, or about 20 molar equivalents of the organic acid, the alkali metal salt thereof, or the combination thereof, relative to the nicotine component, calculated as free base nicotine.

In some embodiments, the composition comprises from about 2 to about 10, or from about 2 to about 5 molar equivalents of the organic acid, the alkali metal salt thereof, or the combination thereof, to nicotine, on a free-base nicotine basis. In some embodiments, the organic acid, the alkali metal salt thereof, or the combination thereof, is present in a molar ratio with the nicotine from about 2, about 3, about 4, or about 5, to about 6, about 7, about 8, about 9, or about 10. In embodiments wherein more than one organic acid, alkali metal salt thereof, or both, are present, it is to be understood that such molar ratios reflect the totality of the organic acids present.

In certain embodiments the organic acid inclusion is sufficient to provide a composition pH of from about 4.0 to about 9.0, such as from about 4.5 to about 7.0, or from about 5.5 to about 7.0, from about 4.0 to about 5.5, or from about 7.0 to about 9.0. In some embodiments, the organic acid inclusion is sufficient to provide a composition pH of from about 4.5 to about 6.5, for example, from about 4.5, about 5.0, or about 5.5, to about 6.0, or about 6.5. In some embodiments, the organic acid is provided in a quantity sufficient to provide a pH of the composition of from about 5.5 to about 6.5, for example, from about 5.5, about 5.6, about 5.7, about 5.8, about 5.9, or about 6.0, to about 6.1, about 6.2, about 6.3, about 6.4, or about 6.5. In other embodiments, a mineral acid (e.g., hydrochloric acid, sulfuric acid, phosphoric acid, or the like) is added to adjust the pH of the composition to the desired value.

In some embodiments, the organic acid is added as the free acid, either neat (i.e., native solid or liquid form) or as a solution in, e.g., water, to the other composition components. In some embodiments, the alkali metal salt of the organic acid is added, either neat or as a solution in, e.g., water, to the other composition components. In some embodiments, the organic acid and the basic amine (e.g., nicotine) are combined to form a salt, either before addition to the composition, or the salt is formed within and is present in the composition as such. In other embodiments, the organic acid and basic amine (e.g., nicotine) are present as individual components in the composition, and form an ion pair upon contact with moisture (e.g., saliva in the mouth of the consumer).

In some embodiments, the composition comprises nicotine benzoate and sodium benzoate (or other alkali metal benzoate). In other embodiments, the composition comprises nicotine and an organic acid, wherein the organic acid is a monoester of a dicarboxylic acid or is a carotenoid derivative having one or more carboxylic acids.

In some embodiments, the composition further comprises a solubility enhancer to increase the solubility of one or more of the organic acid or salt thereof. Suitable solubility enhancers include, but are not limited to, humectants as described herein such as glycerol or propylene glycol.

### Flavoring Agent

In some embodiments, the effervescent composition, the non-effervescent composition, or both the effervescent and the non-effervescent composition as described herein comprises a flavoring agent. As used herein, a “flavoring agent” or “flavorant” is any flavorful or aromatic substance capable of altering the sensory characteristics associated with the oral product. Examples of sensory characteristics that can be modified by the flavoring agent include taste, mouthfeel, moistness, coolness/heat, and/or fragrance/aroma. Flavoring agents may be natural or synthetic, and the character of the flavors imparted thereby may be described, without limitation, as fresh, sweet, herbal, confectionary, floral, fruity, or spicy. Specific types of flavors include, but are not limited to, vanilla, coffee, chocolate/cocoa, cream, mint, spearmint, menthol, peppermint, wintergreen, eucalyptus, lavender, cardamom, nutmeg, cinnamon, clove, cascarilla, sandalwood, honey, jasmine, ginger, anise, sage, licorice, lemon, orange, apple, peach, lime, cherry, strawberry, pineapple, lemon balm, and any combinations thereof. See also, Leffingwell et al., Tobacco Flavoring for Smoking Products, R. J. Reynolds Tobacco Company (1972), which is incorporated herein by reference. Flavorings also may include components that are considered moistening, cooling or smoothening agents, such as eucalyptus. These flavors may be provided neat (i.e., alone) or in a composite, and may be employed as concentrates or flavor packages (e.g., spearmint and menthol, orange and cinnamon; lime, pineapple, and the like). Representative types of components also are set forth in U.S. Pat. No. 5,387,416 to White et al.; U.S. Pat. App. Pub. No. 2005/0244521 to Strickland et al.; and PCT Application Pub. No. WO 05/041699 to Quinter et al., each of which is incorporated herein by reference. In some instances, the flavoring agent may be provided in a spray-dried form or a liquid form.

The amount of flavoring agent utilized can vary, but is typically up to about 10 weight percent, and certain embodiments are characterized by a flavoring agent content of at least about 0.1 weight percent, such as about 0.1 to about 10 weight percent, about 0.3 to about 5 weight percent, or about 0.5 to about 3 weight percent, based on the total dry weight of the individual effervescent and/or non-effervescent composition.

### Sweeteners

In order to improve the sensory properties of the compositions according to the disclosure, one or more sweeteners may be added. The sweeteners can be any sweetener or combination of sweeteners, in natural or artificial form, or as a combination of natural and artificial sweeteners. Examples of natural sweeteners include fructose, sucrose, glucose, maltose, mannose, galactose, lactose, stevia, honey, and the like. Examples of artificial sweeteners include sucralose, isomaltulose, maltodextrin, saccharin, aspartame, acesulfame K, neotame, and the like. In some embodiments, the sweetener comprises one or more sugar alcohols. Sugar alcohols are polyols derived from monosaccharides or disaccharides that have a partially or fully hydrogenated form. Sugar alcohols have, for example, about 4 to about 20 carbon atoms and include erythritol, arabitol, ribitol, isomalt, maltitol, dulcitol, iditol, mannitol, xylitol, lactitol, sorbitol, and combinations thereof (e.g., hydrogenated starch hydrolysates). In some embodiments, the sweetener is xylitol, sucralose, or a combination thereof. In some embodiments, the sweetener is sucralose.

When present, a sweetener or combination of sweeteners may make up from about 0.1 to about 20% or more by weight of the of the effervescent composition, the non-effervescent composition, or both the effervescent composition and the non-effervescent composition, for example, from about 0.1 to about 1%, from about 1 to about 5%, from about 5 to about 10%, or from about 10 to about 20% by weight, based on the total weight of the individual composition(s).

### Taste Modifiers

In order to improve the organoleptic properties of an effervescent and/or non-effervescent composition as disclosed herein, the composition(s) may include one or more taste modifying agents (“taste modifiers”) which may serve to mask, alter, block, or improve e.g., the flavor of the composition(s) as described herein. Non-limiting examples of such taste modifiers include analgesic or anesthetic herbs, spices, and flavors which produce a perceived cooling (e.g., menthol, eucalyptus, mint), warming (e.g., cinnamon), or painful (e.g., capsaicin) sensation. Certain taste modifiers fall into more than one overlapping category.

In some embodiments, the taste modifier modifies one or more of bitter, sweet, salty, or sour tastes. In some embodiments, the taste modifier targets pain receptors. In some embodiments, the effervescent and/or non-effervescent composition comprises an active ingredient having a bitter taste, and a taste modifier which masks or blocks the perception of the bitter taste. In some embodiments, the taste modifier is a substance which targets pain receptors (e.g., vanilloid receptors) in the user's mouth to mask e.g., a bitter taste of another component (e.g., an active ingredient). Suitable taste modifiers include, but are not limited to, capsaicin, gamma-amino butyric acid (GABA), adenosine monophosphate (AMP), lactisole, sodium citrate, or a combination thereof.

When present, a representative amount of taste modifier is about 0.01% by weight or more, about 0.1% by weight or more, or about 1.0% by weight or more, but will typically make up less than about 10% by weight of the total weight of the individual effervescent and/or non-effervescent composition, (e.g., from about 0.01%, about 0.05%, about 0.1%, or about 0.5%, to about 1%, about 5%, or about 10% by weight of the total weight of the individual effervescent and/or non-effervescent composition).

### Salts

In some embodiments, the effervescent and/or non-effervescent composition comprises a salt (e.g., an alkali metal salt), typically employed in an amount sufficient to provide desired sensory attributes to the composition(s). In some embodiments, certain salts may also serve as electrolytes or act in synergy with electrolytes. For example, without wishing to be bound by theory, sodium citrate may provide both a source of sodium (electrolyte) as well as aid in the absorption of other electrolytes and water. Non-limiting examples of suitable salts include sodium chloride, potassium chloride, ammonium chloride, flour salt, sodium acetate, sodium citrate, and the like. In some embodiments, the salt is sodium chloride, ammonium chloride, sodium citrate, or a combination thereof.

When present, a representative amount of salt is about 0.1% by weight or more, about 0.3% by weight or more, or about 0.5% by weight or more, but will typically make up about 10% or less of the total weight of the individual effervescent and/or non-effervescent composition(s), or about 7.5% or less, or about 5% or less (e.g., from about 0.1 to about 5% by weight). In specific embodiments, the effervescent and/or non-effervescent composition comprises sodium citrate in an amount by weight of from about 2 to about 3%, and sodium chloride in an amount by weight of from about 0.1 to about 0.5%, based on the total weight of the individual composition(s).

### Binders

A binder (or combination of binders) may be employed in certain embodiments. Typical binders can be organic or inorganic, or a combination thereof. Representative binders include cellulose derivatives, povidone, sodium alginate, starch-based binders, pectin, carrageenan, pullulan, zein, and the like, and combinations thereof. A binder may be employed in amounts sufficient to provide the desired physical attributes and physical integrity to the effervescent and/or non-effervescent composition. The amount of binder utilized can vary based on the binder and the desired composition properties, but when present, it is typically up to about 30% by weight, and certain embodiments are characterized by a binder content of at least about 0.1% by weight, such as about 0.5 to about 30% by weight, or about 1 to about 20% by weight, based on the total weight of the individual composition(s). In particular embodiments, the effervescent composition is free of binders, and the non-effervescent composition includes a binder.

Suitable binders include cellulose derivatives, such as cellulose ethers (including carboxyalkyl ethers), meaning cellulose polymers with the hydrogen of one or more hydroxyl groups in the cellulose structure replaced with an alkyl, hydroxyalkyl, or aryl group. Non-limiting examples of such cellulose derivatives include methylcellulose, hydroxypropylcellulose (“HPC”), hydroxypropylmethylcellulose (“HPMC”), hydroxyethyl cellulose, and carboxymethylcellulose (“CMC”).

Other suitable binders include a gum, for example, a natural gum. As used herein, a natural gum refers to polysaccharide materials of natural origin that have binding properties, and which are also useful as a thickening or gelling agents. Representative natural gums derived from plants, which are typically water soluble to some degree, include xanthan gum, guar gum, gum arabic, ghatti gum, gum tragacanth, karaya gum, locust bean gum, gellan gum, and combinations thereof. When present, natural gum binder materials are typically present in an amount of up to about 5% by weight, for example, from about 0.1, about 0.2, about 0.3, about 0.4, about 0.5, about 0.6, about 0.7, about 0.8, about 0.9, or about 1%, to about 2, about 3, about 4, or about 5% by weight, based on the total weight of the individual composition(s).

In some embodiments, the non-effervescent composition comprises one or more binders as described herein. In some embodiments, the one or more binders comprise a cellulose derivative, such as a cellulose ether. In some embodiments, the binder is hydroxypropylcellulose. In some embodiments, the binder (e.g., hydroxypropylcellulose) is present in an amount by weight from about 10 to about 20%, based on the total weight of the non-effervescent composition. Without wishing to be bound by theory, it is believed that the presence of a binder in the non-effervescent composition may provide greater cohesiveness to the non-effervescent composition relative to the effervescent composition. In such embodiments, the greater cohesiveness may allow a more gradual dissolution of the layer comprising the non-effervescent composition, thereby providing a sustained or prolonged release of the active ingredient and/or flavor present in the non-effervescent composition.

### Buffering Agents

In certain embodiments, the effervescent and/or non-effervescent composition of the present disclosure can comprise pH adjusters or buffering agents. Examples of pH adjusters and buffering agents that can be used include, but are not limited to, metal hydroxides (e.g., alkali metal hydroxides such as sodium hydroxide and potassium hydroxide), and other alkali metal buffers such as metal carbonates (e.g., potassium carbonate or sodium carbonate), or metal bicarbonates such as sodium bicarbonate, and the like. Non-limiting examples of suitable buffers include alkali metals acetates, glycinates, phosphates, glycerophosphates, citrates, carbonates, hydrogen carbonates, borates, or mixtures thereof.

Where present, the buffering agent is typically present in an amount less than about 5% by weight, based on the weight of the individual effervescent and/or non-effervescent composition, for example, from about 0.1% to about 5%, such as, e.g., from about 0.1% to about 1%, or from about 0.1% to about 0.5% by weight, based on the total weight of the individual composition(s).

### Colorants

A colorant may be employed in amounts sufficient to provide the desired physical attributes to the effervescent and/or non-effervescent composition. Examples of colorants include various dyes and pigments, such as caramel coloring and titanium dioxide. The amount of colorant utilized in the individual composition(s) can vary, but when present is typically up to about 3% by weight, such as from about 0.1%, about 0.5%, or about 1%, to about 3% by weight, based on the total weight of the individual composition(s).

### Humectants

In certain embodiments, one or more humectants may be employed in the effervescent and/or non-effervescent composition. Examples of humectants include, but are not limited to, glycerin, propylene glycol, and the like. Where included, the humectant is typically provided in an amount sufficient to provide desired moisture attributes to the composition(s). Further, in some instances, the humectant may impart desirable flow characteristics to the effervescent and/or non-effervescent composition, e.g., for depositing in a mold.

When present, a humectant will typically make up about 5% or less of the weight of the individual composition(s) (e.g., from about 0.1 to about 5% by weight), for example, from about 0.1% to about 1% by weight, or about 1% to about 5% by weight, based on the total weight of the individual composition.

### Tobacco Material

In some embodiments, the effervescent and/or non-effervescent composition may include a tobacco material. The tobacco material can vary in species, type, and form. Generally, the tobacco material is obtained from for a harvested plant of the Nicotiana species. Example Nicotiana species include N. tabacum, N. rustica, N. alata, N. arentsii, N. excelsior, N. forgetiana, N. glauca, N. glutinosa, N. gossei, N. kawakamii, N. knightiana, N. langsdorffi, N. otophora, N. setchelli, N. sylvestris, N. tomentosa, N. tomentosiformis, N. undulata, N. x sanderae, N. africana, N. amplexicaulis, N. benavidesii, N. bonariensis, N. debneyi, N. longiflora, N. maritina, N. megalosiphon, N. occidentalis, N. paniculata, N. plumbaginifolia, N. raimondii, N. rosulata, N. simulans, N. stocktonii, N. suaveolens, N. umbratica, N. velutina, N. wigandioides, N. acaulis, N. acuminata, N. attenuata, N. benthamiana, N. cavicola, N. clevelandii, N. cordifolia, N. corymbosa, N. fragrans, N. goodspeedii, N. linearis, N. miersii, N. nudicaulis, N. obtusifolia, N. occidentalis subsp. Hersperis, N. pauciflora, N. petunioides, N. quadrivalvis, N. repanda, N. rotundifolia, N. solanifolia, and N. spegazzinii Various representative other types of plants from the Nicotiana species are set forth in Goodspeed, The Genus Nicotiana, (Chonica Botanica) (1954); U.S. Pat. No. 4,660,577 to Sensabaugh, Jr. et al.; 5,387,416 to White et al., U.S. Pat. No. 7,025,066 to Lawson et al.; U.S. Pat. No. 7,798,153 to Lawrence, Jr. and 8,186,360 to Marshall et al.; each of which is incorporated herein by reference. Descriptions of various types of tobaccos, growing practices and harvesting practices are set forth in Tobacco Production, Chemistry and Technology, Davis et al. (Eds.) (1999), which is incorporated herein by reference.

Nicotiana species from which suitable tobacco materials can be obtained can be derived using genetic-modification or crossbreeding techniques (e.g., tobacco plants can be genetically engineered or crossbred to increase or decrease production of components, characteristics or attributes). See, for example, the types of genetic modifications of plants set forth in U.S. Pat. No. 5,539,093 to Fitzmaurice et al.; U.S. Pat. No. 5,668,295 to Wahab et al.; U.S. Pat. No. 5,705,624 to Fitzmaurice et al.; U.S. Pat. No. 5,844,119 to Weigl; U.S. Pat. No. 6,730,832 to Dominguez et al.; U.S. Pat. No. 7,173,170 to Liu et al.; U.S. Pat. No. 7,208,659 to Colliver et al. and 7,230,160 to Benning et al.; US Patent Appl. Pub. No. 2006/0236434 to Conkling et al.; and PCT WO2008/103935 to Nielsen et al. See, also, the types of tobaccos that are set forth in U.S. Pat. No. 4,660,577 to Sensabaugh, Jr. et al.; 5,387,416 to White et al.; and 6,730,832 to Dominguez et al., each of which is incorporated herein by reference.

The Nicotiana species can, in some embodiments, be selected for the content of various compounds that are present therein. For example, plants can be selected on the basis that those plants produce relatively high quantities of one or more of the compounds desired to be isolated therefrom. In certain embodiments, plants of the Nicotiana species (e.g., Galpao commun tobacco) are specifically grown for their abundance of leaf surface compounds. Tobacco plants can be grown in greenhouses, growth chambers, or outdoors in fields, or grown hydroponically.

Various parts or portions of the plant of the Nicotiana species can be included within a composition as disclosed herein. For example, virtually all of the plant (e.g., the whole plant) can be harvested, and employed as such. Alternatively, various parts or pieces of the plant can be harvested or separated for further use after harvest. For example, the flower, leaves, stem, stalk, roots, seeds, and various combinations thereof, can be isolated for further use or treatment. In some embodiments, the tobacco material comprises tobacco leaf (lamina). The compositions disclosed herein can include processed tobacco parts or pieces, cured and aged tobacco in essentially natural lamina and/or stem form, a tobacco extract, extracted tobacco pulp (e.g., using water as a solvent), or a mixture of the foregoing (e.g., a mixture that combines extracted tobacco pulp with granulated cured and aged natural tobacco lamina).

In certain embodiments, the tobacco material comprises solid tobacco material selected from the group consisting of lamina and stems. The tobacco that is used for the mixture most preferably includes tobacco lamina, or a tobacco lamina and stem mixture (of which at least a portion is smoke-treated). Portions of the tobaccos within the mixture may have processed forms, such as processed tobacco stems (e.g., cut-rolled stems, cut-rolled-expanded stems or cut-puffed stems), or volume expanded tobacco (e.g., puffed tobacco, such as dry ice expanded tobacco (DIET)). See, for example, the tobacco expansion processes set forth in U.S. Pat. No. 4,340,073 to de la Burde et al.; U.S. Pat. No. 5,259,403 to Guy et al.; and 5,908,032 to Poindexter, et al.; and 7,556,047 to Poindexter, et al., all of which are incorporated by reference. In addition, the d mixture optionally may incorporate tobacco that has been fermented. See, also, the types of tobacco processing techniques set forth in PCT WO2005/063060 to Atchley et al., which is incorporated herein by reference.

The tobacco material is typically used in a form that can be described as particulate (i.e., shredded, ground, granulated, or powder form). The manner by which the tobacco material is provided in a finely divided or powder type of form may vary. Preferably, plant parts or pieces are comminuted, ground or pulverized into a particulate form using equipment and techniques for grinding, milling, or the like. Most preferably, the plant material is relatively dry in form during grinding or milling, using equipment such as hammer mills, cutter heads, air control mills, or the like. For example, tobacco parts or pieces may be ground or milled when the moisture content thereof is less than about 15% by weight, or less than about % by weight. Most preferably, the tobacco material is employed in the form of parts or pieces that have an average particle size between 1.4 millimeters and 250 microns. In some instances, the tobacco particles may be sized to pass through a screen mesh to obtain the particle size range required. If desired, air classification equipment may be used to ensure that small sized tobacco particles of the desired sizes, or range of sizes, may be collected. If desired, differently sized pieces of granulated tobacco may be mixed together.

The manner by which the tobacco is provided in a finely divided or powder type of form may vary. Preferably, tobacco parts or pieces are comminuted, ground or pulverized into a powder type of form using equipment and techniques for grinding, milling, or the like. Most preferably, the tobacco is relatively dry in form during grinding or milling, using equipment such as hammer mills, cutter heads, air control mills, or the like. For example, tobacco parts or pieces may be ground or milled when the moisture content thereof is less than about 15% by weight to less than about 5% by weight. For example, the tobacco plant or portion thereof can be separated into individual parts or pieces (e.g., the leaves can be removed from the stems, and/or the stems and leaves can be removed from the stalk). The harvested plant or individual parts or pieces can be further subdivided into parts or pieces (e.g., the leaves can be shredded, cut, comminuted, pulverized, milled or ground into pieces or parts that can be characterized as filler-type pieces, granules, particulates or fine powders). The plant, or parts thereof, can be subjected to external forces or pressure (e.g., by being pressed or subjected to roll treatment). When carrying out such processing conditions, the plant or portion thereof can have a moisture content that approximates its natural moisture content (e.g., its moisture content immediately upon harvest), a moisture content achieved by adding moisture to the plant or portion thereof, or a moisture content that results from the drying of the plant or portion thereof. For example, powdered, pulverized, ground or milled pieces of plants or portions thereof can have moisture contents of less than about 25% by weight, often less than about 20%, and frequently less than about 15% by weight.

For the preparation of tobacco-containing compositions, it is typical for a harvested plant of the Nicotiana species to be subjected to a curing process. The tobacco materials incorporated within the composition(s) as disclosed herein are those that have been appropriately cured and/or aged. Descriptions of various types of curing processes for various types of tobaccos are set forth in Tobacco Production, Chemistry and Technology, Davis et al. (Eds.) (1999). Examples of techniques and conditions for curing flue-cured tobacco are set forth in Nestor et al., Beitrage Tabakforsch. Int., 20, 467-475 (2003) and U.S. Pat. No. 6,895,974 to Peele, which are incorporated herein by reference. Representative techniques and conditions for air curing tobacco are set forth in U.S. Pat. No. 7,650,892 to Groves et al.; Roton et al., Beitrage Tabakforsch. Int., 21, 305-320 (2005) and Staaf et al., Beitrage Tabakforsch. Int., 21, 321-330 (2005), which are incorporated herein by reference. Certain types of tobaccos can be subjected to alternative types of curing processes, such as fire curing or sun curing.

In certain embodiments, tobacco materials that can be employed include flue-cured or Virginia (e.g., K326), burley, sun-cured (e.g., Indian Kurnool and Oriental tobaccos, including Katerini, Prelip, Komotini, Xanthi and Yambol tobaccos), Maryland, dark, dark-fired, dark air cured (e.g., Madole, Passanda, Cubano, Jatin and Bezuki tobaccos), light air cured (e.g., North Wisconsin and Galpao tobaccos), Indian air cured, Red Russian and Rustica tobaccos, as well as various other rare or specialty tobaccos and various blends of any of the foregoing tobaccos.

The tobacco material may also have a so-called “blended” form. For example, the tobacco material may include a mixture of parts or pieces of flue-cured, burley (e.g., Malawi burley tobacco) and Oriental tobaccos (e.g., as tobacco composed of, or derived from, tobacco lamina, or a mixture of tobacco lamina and tobacco stem). For example, a representative blend may incorporate about 30 to about 70 parts burley tobacco (e.g., lamina, or lamina and stem), and about 30 to about 70 parts flue cured tobacco (e.g., stem, lamina, or lamina and stem) on a dry weight basis. Other example tobacco blends incorporate about 75 parts flue-cured tobacco, about 15 parts burley tobacco, and about 10 parts Oriental tobacco; or about 65 parts flue-cured tobacco, about 25 parts burley tobacco, and about 10 parts Oriental tobacco; or about 65 parts flue-cured tobacco, about 10 parts burley tobacco, and about 25 parts Oriental tobacco; on a dry weight basis. Other example tobacco blends incorporate about 20 to about 30 parts Oriental tobacco and about 70 to about 80 parts flue-cured tobacco on a dry weight basis.

Tobacco materials used in the present disclosure can be subjected to, for example, fermentation, bleaching, and the like. If desired, the tobacco materials can be, for example, irradiated, pasteurized, or otherwise subjected to controlled heat treatment. Such treatment processes are detailed, for example, in U.S. Pat. No. 8,061,362 to Mua et al., which is incorporated herein by reference. In certain embodiments, tobacco materials can be treated with water and an additive capable of inhibiting reaction of asparagine to form acrylamide upon heating of the tobacco material (e.g., an additive selected from the group consisting of lysine, glycine, histidine, alanine, methionine, cysteine, glutamic acid, aspartic acid, proline, phenylalanine, valine, arginine, compositions incorporating di- and trivalent cations, asparaginase, certain non-reducing saccharides, certain reducing agents, phenolic compounds, certain compounds having at least one free thiol group or functionality, oxidizing agents, oxidation catalysts, natural plant extracts (e.g., rosemary extract), and combinations thereof. See, for example, the types of treatment processes described in U.S. Pat. Pub. Nos. 8,434,496, 8,944,072, and 8,991,403 to Chen et al., which are all incorporated herein by reference. In certain embodiments, this type of treatment is useful where the original tobacco material is subjected to heat in the processes previously described.

In various embodiments, the tobacco material can be treated to extract a soluble component of the tobacco material therefrom. “Tobacco extract” as used herein refers to the isolated components of a tobacco material that are extracted from solid tobacco pulp by a solvent that is brought into contact with the tobacco material in an extraction process. Various extraction techniques of tobacco materials can be used to provide a tobacco extract and tobacco solid material. See, for example, the extraction processes described in U.S. Pat. Appl. Pub. No. 2011/0247640 to Beeson et al., which is incorporated herein by reference. Other example techniques for extracting components of tobacco are described in U.S. Pat. No. 4,144,895 to Fiore; U.S. Pat. No. 4,150,677 to Osborne, Jr. et al.; 4,267,847 to Reid; U.S. Pat. No. 4,289,147 to Wildman et al.; U.S. Pat. No. 4,351,346 to Brummer et al.; U.S. Pat. No. 4,359,059 to Brummer et al.; U.S. Pat. No. 4,506,682 to Muller; U.S. Pat. No. 4,589,428 to Keritsis; U.S. Pat. No. 4,605,016 to Soga et al.; U.S. Pat. No. 4,716,911 to Poulose et al.; U.S. Pat. No. 4,727,889 to Niven, Jr. et al.; 4,887,618 to Bernasek et al.; U.S. Pat. No. 4,941,484 to Clapp et al.; U.S. Pat. No. 4,967,771 to Fagg et al.; U.S. Pat. No. 4,986,286 to Roberts et al.; U.S. Pat. No. 5,005,593 to Fagg et al.; U.S. Pat. No. 5,018,540 to Grubbs et al.; U.S. Pat. No. 5,060,669 to White et al.; U.S. Pat. No. 5,065,775 to Fagg; U.S. Pat. No. 5,074,319 to White et al.; U.S. Pat. No. 5,099,862 to White et al.; U.S. Pat. No. 5,121,757 to White et al.; U.S. Pat. No. 5,131,414 to Fagg; U.S. Pat. No. 5,131,415 to Munoz et al.; U.S. Pat. No. 5,148,819 to Fagg; U.S. Pat. No. 5,197,494 to Kramer; U.S. Pat. No. 5,230,354 to Smith et al.; U.S. Pat. No. 5,234,008 to Fagg; U.S. Pat. No. 5,243,999 to Smith; U.S. Pat. No. 5,301,694 to Raymond et al.; U.S. Pat. No. 5,318,050 to Gonzalez-Parra et al.; U.S. Pat. No. 5,343,879 to Teague; U.S. Pat. No. 5,360,022 to Newton; U.S. Pat. No. 5,435,325 to Clapp et al.; U.S. Pat. No. 5,445,169 to Brinkley et al.; U.S. Pat. No. 6,131,584 to Lauterbach; U.S. Pat. No. 6,298,859 to Kierulff et al.; U.S. Pat. No. 6,772,767 to Mua et al.; and 7,337,782 to Thompson, all of which are incorporated by reference herein.

In some embodiments, the type of tobacco material is selected such that it is initially visually lighter in color than other tobacco materials to some degree (e.g., whitened or bleached). Tobacco pulp can be whitened in certain embodiments according to any means known in the art, and as described above in reference to color-eliminated active ingredients.

Typical inclusion ranges for tobacco materials can vary depending on the nature and type of the tobacco material, and the intended effect on the final effervescent and/or non-effervescent composition, with an example range of up to about 30% by weight (or up to about 20% by weight or up to about 10% by weight or up to about 5% by weight), based on total weight of the individual composition (e.g., about 0.1 to about 15% by weight). In some embodiments, the products of the disclosure can be characterized as completely free or substantially free of tobacco material (other than purified nicotine as an active ingredient). For example, certain embodiments can be characterized as having less than 1% by weight, or less than 0.5% by weight, or less than 0.1% by weight of tobacco material, or 0% by weight of tobacco material.

### Oral Care Additives

In some embodiments, the effervescent and/or non-effervescent composition comprises an oral care ingredient (or mixture of such ingredients). Oral care ingredients provide the ability to inhibit tooth decay or loss, inhibit gum disease, relieve mouth pain, whiten teeth, or otherwise inhibit tooth staining, elicit salivary stimulation, inhibit breath malodor, freshen breath, or the like. For example, effective amounts of ingredients such as thyme oil, eucalyptus oil and zinc (e.g., such as the ingredients of formulations commercially available as ZYTEX® from Discus Dental) can be incorporated into the effervescent composition. Other examples of ingredients that can be incorporated in desired effective amounts within the present compositions can include those that are incorporated within the types of oral care compositions set forth in Takahashi et al., Oral Microbiology and Immunology, 19(1), 61-64 (2004); U.S. Pat. No. 6,083,527 to Thistle; and U.S. Pat. Appl. Pub. Nos. 2006/0210488 to Jakubowski and 2006/02228308 to Cummins et al. Other exemplary ingredients of tobacco containing-formulation include those contained in formulations marketed as MALTISORB® by Roquette and DENTIZYME® by NatraRx. When present, a representative amount of oral care additive is at least about 1%, often at least about 3%, and frequently at least about 5% of the total dry weight of the individual composition. The amount of oral care additive within the individual composition will not typically exceed about 30%, often will not exceed about 25%, and frequently will not exceed about 20%, of the total dry weight of the individual composition.

### Processing Aids

If necessary for downstream processing of the effervescent and/or non-effervescent composition, such as granulation, mixing, or molding, a flow aid or lubricant can also be added to the composition(s) in order to enhance flowability. Exemplary flow aids and lubricants include microcrystalline cellulose, silica, polyethylene glycol, stearic acid, calcium stearate, magnesium stearate, zinc stearate, sodium stearyl fumarate, canauba wax, and combinations thereof. In some embodiments, the flow aid is silica, stearic acid, magnesium stearate, or a combination thereof. In some embodiments, the flow aid is sodium stearyl fumarate.

When present, a representative amount of flow aid may make up at least about 0.5 percent or at least about 1 percent, of the total dry weight of the individual composition. Preferably, the amount of flow aid within the individual composition will not exceed about 5 percent, and frequently will not exceed about 3 percent, of the total dry weight of the individual composition.

### Emulsifier

In certain embodiments, an emulsifier may be added. In certain embodiments, lecithin can be added to the effervescent and/or non-effervescent composition to provide smoother textural properties and to improve flowability and mixing of components of the compositions. Lecithin can be used in an amount of about 0.01 to about 5% by dry weight of the individual effervescent and/or non-effervescent composition, such as about 0.1 to about 2.5% or about 0.1 to about 1.0%.

### Other Additives

Other additives can be included in the disclosed compositions. For example, the effervescent and/or non-effervescent composition can be processed, blended, formulated, combined, and/or mixed with other materials or ingredients. The additives can be artificial, or can be obtained or derived from herbal or biological sources. Examples of further types of additives include thickening or gelling agents (e.g., fish gelatin), emulsifiers, preservatives (e.g., potassium sorbate and the like), disintegration aids, or combinations thereof. See, for example, those representative components, combination of components, relative amounts of those components, and manners and methods for employing those components, set forth in U.S. Pat. No. 9,237,769 to Mua et al., U.S. Pat. No. 7,861,728 to Holton, Jr. et al., U.S. Pat. App. Pub. No. 2010/0291245 to Gao et al., and US Pat. App. Pub. No. 2007/0062549 to Holton, Jr. et al., each of which is incorporated herein by reference. Typical inclusion ranges for such additional additives can vary depending on the nature and function of the additive and the intended effect on the final composition, with an example range of up to about 10% by weight, based on total weight of the individual effervescent and/or non-effervescent composition, (e.g., about 0.1 to about 5% by weight).

The aforementioned additives can be employed together (e.g., as additive formulations) or separately (e.g., individual additive components can be added at different stages involved in the preparation of the final composition). Furthermore, the aforementioned types of additives may be encapsulated as provided in the final effervescent and/or non-effervescent composition. Example encapsulated additives are described, for example, in WO2010/132444 to Atchley, which has been previously incorporated by reference herein.

### Configured for Oral Use

The products (e.g., multi-layered tablets) provided herein are configured for oral use. The term “configured for oral use” as used herein means that the product is provided in a form such that during use, saliva in the mouth of the user causes components of the compositions therein (e.g., flavoring agents and/or active ingredients) to pass into the mouth of the user. Generally, saliva in the mouth of the user causes the effervescent material in the effervescent composition to effervesce in the oral cavity. In certain embodiments, the effervescent composition is adapted to deliver components to a user through mucous membranes in the user's mouth, the user's digestive system, or both, and, in some instances, said component is an active ingredient (including, but not limited to, for example, a stimulant) that can be absorbed through the mucous membranes in the mouth or absorbed through the digestive tract when the product is used. Similarly, the non-effervescent composition is adapted to deliver components to a user through mucous membranes in the user's mouth, the user's digestive system, or both, and, in some instances, said component is an active ingredient (including, but not limited to, for example, a stimulant) that can be absorbed through the mucous membranes in the mouth or absorbed through the digestive tract when the product is used.

Certain products can exhibit, for example, one or more of the following characteristics: crispy, granular, chewy, syrupy, pasty, fluffy, smooth, and/or creamy. In certain embodiments, the desired textural property can be selected from the group consisting of adhesiveness, cohesiveness, density, dryness, fracturability, graininess, gumminess, hardness, heaviness, moisture absorption, moisture release, mouthcoating, roughness, slipperiness, smoothness, viscosity, wetness, and combinations thereof.

The effervescent and/or non-effervescent compositions of the present disclosure may be meltable as discussed, for example, in US Patent App. Pub. No. 2012/0037175 to Cantrell et al. As used herein, “melt,” “melting,” and “meltable” refer to the ability of the composition to change from a solid state to a liquid state. That is, melting occurs when a substance (e.g., a composition as disclosed herein) changes from solid to liquid, usually by the application of heat. The application of heat in regard to a composition as disclosed herein is provided by the internal temperature of a user's mouth. Thus, the term “meltable” refers to a composition that is capable of liquefying in the mouth of the user as the composition changes phase from solid to liquid, and is intended to distinguish compositions that merely disintegrate in the oral cavity through loss of cohesiveness within the composition that merely dissolve in the oral cavity as aqueous-soluble components of the composition interact with moisture. Generally, meltable compositions comprise a lipid as described herein above.

The effervescent and/or non-effervescent compositions of the present disclosure may be dissolvable. As used herein, the terms “dissolve,” “dissolving,” “dissolvable” are used interchangeably, and refer to compositions having aqueous-soluble components that interact with moisture in the oral cavity and enter into solution, thereby causing gradual consumption of the product. According to one aspect, a dissolvable product is capable of lasting in the user's mouth for a given period of time until it completely dissolves. Dissolution rates can vary over a wide range, from about 1 minute or less to about 60 minutes. For example, fast release effervescent compositions typically dissolve and/or release the active substance in about 2 minutes or less, often about 1 minute or less (e.g., about 50 seconds or less, about 40 seconds or less, about 30 seconds or less, or about 20 seconds or less). Dissolution can occur by any means, such as melting, mechanical disruption (e.g., chewing), enzymatic or other chemical degradation, or by disruption of the interaction between the components of the effervescent composition. In other embodiments, the products do not dissolve during the product's residence in the user's mouth.

The products configured for oral use can be formed into a variety of shapes, including pills, tablets, spheres, strips, films, sheets, coins, cubes, beads, ovoids, obloids, cylinders, bean-shaped, sticks, or rods. Cross-sectional shapes of the product can vary, and example cross-sectional shapes include circles, squares, ovals, rectangles, and the like. Such shapes can be formed in a variety of manners using equipment such as moving belts, nips, extruders, granulation devices, compaction devices, and the like.

As described herein above, in some embodiments is provided a compressed or molded multi-layered tablet, wherein a first layer comprises an effervescent composition as described herein, and the second layer comprises a non-effervescent composition as described herein. Such embodiments may be configured to independently provide a first and a second active ingredient, a first and second flavoring agent, or both to the user during use of the tablet. The first and second active ingredients or flavoring agents may be the same or different. In some embodiments, the multi-layered tablet is configured to provide the first active ingredient rapidly, and to provide the second active ingredient more gradually. Such embodiments are described further below with respect to the preparation thereof. Alternatively, in some embodiments, the multi-layer tablet is free of active ingredients, and is configured to provide a first flavoring agent rapidly, and to provide a second flavoring agent more gradually.

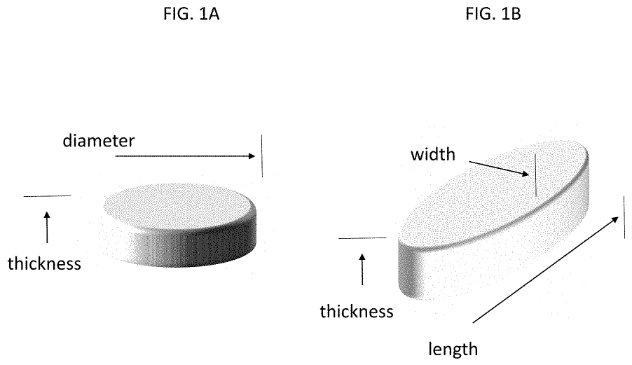

The compressed or molded multi-layered tablet can have any of a variety of shapes, including traditional round or ovoid tablet shapes. Certain embodiments of the disclosure will be described with reference to FIG. 1A and FIG. 1B, in which non-limiting examples of possible tablet shapes are provided. Referring to FIG. 1A, there is shown in a perspective view an embodiment of a tablet comprising the effervescent and non-effervescent compositions in the form of a tablet having a diameter and a thickness. Referring to FIG. 1B, there is shown in a perspective view an embodiment of a tablet comprising the effervescent and non-effervescent composition, the tablet having an ovoid shape having a length, a width, and a thickness.

The precise shape and size of such tablets is immaterial, but generally, a tablet will have a length, width, and thickness, or a diameter and thickness, which provide a relatively large surface area. The dimensions will vary based on the weight of the tablet. Example tablet weights range from about 250 mg to about 1500 mg, such as about 250 mg to about 700 mg, or from about 700 mg to about 1500 mg. Example tablet sizes include tablets having a length and width in the range of about 3 mm to about 20 mm, and more typically from about 5 to about 18 mm. Example tablet sizes include tablets having a thickness in the range of about 3 to about 10 mm. In some embodiments, the tablet has a length of from about 15 mm to about 20 mm, a width of from about 6 to about 10 mm, and a thickness of from about 3 to about 6 mm. In a particular embodiment, the length is about 18 mm, the width is about 9 mm, and the thickness is about 5 mm. In some embodiments, the tablet has a diameter of from about 9 mm to about 20 mm, and a thickness of from about 3 to about 10 mm, or from about 4 to about 6 mm.

In some embodiments, the effervescent composition is in the form of a tablet of ovoid or obloid shape having a length and width, each of which is greater than the thickness. Such embodiments may be described in terms of an aspect ratio, defined herein as the ratio of the smallest dimension to the largest dimension. In some embodiments, the effervescent composition is in the form of a tablet having an aspect ratio of from about 1.5 to about 3. In certain embodiments, it may be advantageous to select parameters such as aspect ratio, and the associated thickness, width, length, or diameter, to provide a product having a high surface area. Without wishing to be bound by theory, it is believed that high surface area products, by exposing more surface area to moisture (e.g., the saliva present in the mouth of the consumer), allow for a favorable rate of effervescence, which may result in a positive consumer experience.

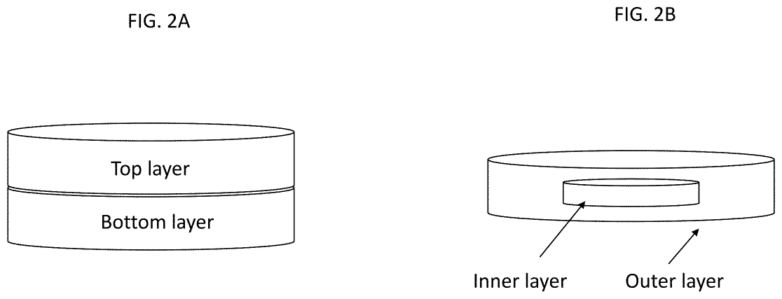

Referring to FIG. 2A, there is shown in a perspective view a non-limiting embodiment of a multi-layer tablet having two layers; a top layer and a bottom layer, in which at least one of the layers comprises the effervescent composition. Referring to FIG. 2B, there is shown in a perspective view a non-limiting embodiment of a tablet having an inner layer and an outer layer, in which at least one of the layers comprises the effervescent composition. The particular arrangement and number of layers depicted is for illustration only and are non-limiting, and other arrangements (number of layers and configuration thereof) are contemplated by the present disclosure.

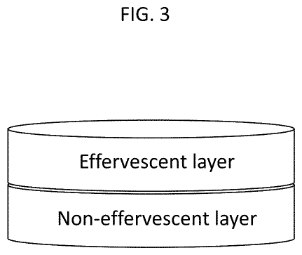

Referring to FIG. 3, there is shown in a perspective view another non-limiting embodiment of a multi-layer tablet having two layers; a first layer and a second layer, in which the first (top) layer comprises an effervescent composition as disclosed herein, and the second (bottom) layer comprises a non-effervescent composition as described herein. Beyond effervescence, the first and second layers may have further differential properties, such as different dissolution rates, different textures, different active ingredients, different flavoring agents, or different textural properties. In particular embodiments, the effervescent layer is configured to provide rapid release of an active ingredient and/or flavorant, and the non-effervescent layer is configured to provide a gradual release of an active ingredient and/or flavorant. In such embodiments, the non-effervescent layer may comprise a component which enhances the cohesive properties of the non-effervescent composition (e.g., a binder), thereby attenuating the release of the active ingredient and/or flavorant from the non-effervescent layer upon use of the tablet. Non-limiting examples of suitable binders include cellulose derivatives, such as cellulose ethers. In some embodiments, the two-layer tablet provides an initial rapid release of an active ingredient, followed by a slower and longer lasting release of an active agent. The two active ingredients may be the same or different. In some embodiments, the two active ingredients are different. In some embodiments, the two active ingredients are the same. In some embodiments, the same two active ingredients are present in different concentrations in the two layers. In some embodiments, the same two active ingredients are present in the same concentrations in both layers. In some embodiments, both active ingredients are a nicotine component as described herein. In some embodiments, the nicotine component is present in the same concentration in each layer.

### Preparation of the Compositions and Tablets

The manner by which the various components of the effervescent compositions (e.g., effervescent material, filler, active ingredient, flavoring agent, and optionally, a lipid) are combined may vary. As such, the overall effervescent composition with e.g., powdered composition components may be relatively uniform in nature. The components noted above, which may be in liquid or dry solid form, can be admixed in a pretreatment step prior to mixture with any remaining components of the composition, or simply mixed together with all other liquid or dry ingredients.

The effervescent compositions of the disclosure are prepared, for example, by dry-blending dry ingredients, such as filler, sweeteners, salts, and the like. In certain embodiments, water can be added to the dry blend at this stage. Additionally, it is optional to add, such as by spraying, active ingredients and/or flavoring agents to the dry blend, followed by mixing.

The various components of the effervescent composition may be contacted, combined, or mixed together using any mixing technique or equipment known in the art. Any mixing method that brings the effervescent composition ingredients into intimate contact can be used, such as a mixing apparatus featuring an impeller or other structure capable of agitation. Examples of mixing equipment include casing drums, conditioning cylinders or drums, liquid spray apparatus, conical-type blenders, ribbon blenders, mixers available as FKM130, FKM600, FKM1200, FKM2000 and FKM3000 from Littleford Day, Inc., Plough Share types of mixer cylinders, Hobart mixers, and the like. See also, for example, the types of methodologies set forth in U.S. Pat. No. 4,148,325 to Solomon et al.; U.S. Pat. No. 6,510,855 to Korte et al.; and 6,834,654 to Williams, each of which is incorporated herein by reference. In some embodiments, the components forming the effervescent composition are prepared such that the mixture thereof may be used in a starch molding process for forming the effervescent composition. Manners and methods for formulating effervescent compositions will be apparent to those skilled in the art. See, for example, the types of methodologies set forth in U.S. Pat. No. 4,148,325 to Solomon et al.; U.S. Pat. No. 6,510,855 to Korte et al.; and U.S. Pat. No. 6,834,654 to Williams, U.S. Pat. No. 4,725,440 to Ridgway et al., and 6,077,524 to Bolder et al., each of which is incorporated herein by reference.

In some embodiments, the effervescent composition is in the form of a tablet. Compressed effervescent composition tablets can be produced by compacting the effervescent composition, including any associated formulation components, in the form of a tablet, and optionally coating each tablet with an overcoat material.

The effervescent composition can include an optional outer coating, which can help to improve storage stability of the product as well as improve the packaging process by reducing friability and dusting. The coating typically comprises a film-forming polymer, such as a cellulosic polymer, an optional plasticizer, and optional flavorants, colorants, salts, sweeteners or other additives of the types set forth herein. The coating compositions are usually aqueous in nature and can be applied using any pellet or tablet coating technique known in the art, such as pan coating. Example film-forming polymers include cellulosic polymers such as methylcellulose, hydroxypropyl cellulose (HPC), hydroxypropyl methylcellulose (HPMC), hydroxyethyl cellulose, and carboxy methylcellulose. Example plasticizers include aqueous solutions or emulsions of glyceryl monostearate and triethyl citrate.

In one embodiment, the process for making the effervescent composition in the form of a tablet involves first forming a granulation mixture, which may be tobacco-containing, granulating the mixture, optionally adding a binder, or a solution thereof, to produce an intermediate granular product, and then blending the granules with a second composition comprising the additional effervescent composition components to form the final effervescent composition. The final effervescent composition is then compressed into pellet or tablet form and optionally coated. The granulation mixture typically includes a first portion of the acid component of the effervescent material (e.g., a first portion of a mixture of citric acid and tartaric acid), optionally a first portion of the base component of the effervescent material (e.g., a carbonate material), and optionally one or more binders, fillers, lipids, sweeteners, flavorants, active ingredients, colorants, compressibility aids, or other additives. It is desirable to maintain the effervescent composition in a relatively inert state during manufacture so that the effervescing effect is preserved in the final product. Bicarbonate base materials are more reactive with an acid to create effervescence in the presence of moisture and therefore can lead to premature reactivity in the product. The granulation mixture is typically relatively dry, meaning no liquid ingredients are introduced and instead the mixture contains essentially all dry powder ingredients. The granulation material may be mixed with a binder solution (e.g., by spraying the binder solution into the granulator) and granulated to a desired particle size, such as about 100 to about 200 microns. As would be understood in the art, the binder solution facilitates agglomeration of the dry powder granulation mixture into larger granules.

The binder solution used in the granulation process can be any aqueous or alcohol-based solution containing a binding agent, particularly a polymeric binding agent such as povidone or hydroxypropylcellulose, and can contain other additives including any of the additives discussed herein such as mannitol, maltodextrin, tobacco material, sweeteners, flavorants, and effervescent materials. The binder solution will typically have a solids content of about 5 to about 20 percent (w/w), and preferred solvents include water and ethanol. The binder solution used in the granulation process can be aqueous in nature without causing significant premature effervescence within the granulation mixture.

Following granulation, the granules are advantageously dried, typically to a moisture level of less than about 7.0 weight percent, more typically less than about 6.5 weight percent, and often less than about 6.0 weight percent (e.g., a range of about 4.0 to about 7.0 weight percent). An exemplary moisture level is about 5.5 weight percent.

The dried granules are then blended with the remaining desired components of the effervescent composition, including for example, a second portion of the acid component of the effervescent material (e.g., a second portion of a mixture of citric acid and tartaric acid), a base component of the effervescent material (e.g., a bicarbonate material), and optionally one or more binders, fillers, lipids, sweeteners, flavorants, colorants, flow aids, or other additives. The blending of the granulated material with the remaining ingredients can be accomplished using a granulator or any other mixing device. The final blended material may then be compressed using conventional tableting techniques.

Example granulation devices are available as the FL-M Series granulator equipment (e.g., FL-M-3) from Vector Corporation and as WP 120V and WP 200VN from Alexanderwerk, Inc. Example compaction devices, such as compaction presses, are available as Colton 2216 and Colton 2247 from Vector Corporation and as 1200i, 2200i, 3200, 2090, 3090 and 4090 from Fette Compacting. Devices for providing outer coating layers to compacted pelletized compositions are available as CompuLab 24, CompuLab 36, Accela-Cota 48 and Accela-Cota 60 from Thomas Engineering.

The hardness of the effervescent composition of the disclosure can vary, but is typically at least about 5 kp (kiloponds), such as at least about 8 kp, at least about 10 kp, or at least about 12 kp (e.g., a hardness range of about 5 kp to about 20 kp or about 8 kp to about 15 kp). Hardness can be measured using a hardness tester such as a Varian VK 200 or equivalent.

The non-effervescent compositions may be similarly prepared, albeit in the absence of the effervescent material. The non-effervescent composition may be compressed to form a tablet, and the non-effervescent tablet adhered to the effervescent tablet to form the layered structure.

Other methods of preparing multi-layered products could also be used. For example, a conventional tablet press could be used to manufacture a layered product by simply adding multiple distinct granular compositions to the tablet press. The individual layers can be added by introducing a granular mixture of the desired compositions into a tablet press mold, and a tablet press can be used to compress the granular mixtures to create a layered structure.

In one embodiment, a multi-layer tablet is formed by adding a granular mixture comprising a first composition to the tablet press mold followed by addition of a granular mixture containing a second composition different from the first, either or both of which may be effervescent. This process could be repeated until the desired number of layers is reached. Thereafter, applying pressure to the tablet press mold will result in a pellet or tablet product with multiple, distinct layers. Multi-layered products made using this process could possess the same characteristics as described above in connection with rotor granulation systems. For instance, the pressed tablet could contain multiple effervescent and non-effervescent layers.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

### EXAMPLES

Aspects of the present invention are more fully illustrated by the following examples, which are set forth to illustrate certain aspects of the present invention and are not to be construed as limiting thereof.

### Example 1. Effervescent Tablets Containing Caffeine

Effervescent tablets according to an embodiment of the disclosure were prepared including caffeine as the active ingredient. The dry blend formulation is provided in Table 2. The dry materials (mannitol, maltodextrin, sweetener, caffeine, salt, silicon dioxide, and a pre-formed mixture of sodium bicarbonate, citric acid, and tartaric acid) were each passed through an 18 mesh screen, then mixed in a V-blender until homogenous. Following the mixing period, a punch lubricant (e.g., stearic acid, magnesium stearate, silica/, sodium stearyl fumarate, or combinations thereof) was added as necessary for processing, followed by further mixing. Tablets were prepared using a punch press, forming tablets weighing about 800 mg or about 1000 mg each. The tablets were roughly ovoid in shape, having a length of approximately 18 mm and a width of approximately 9 mm, and with a thickness of approximately 6 mm.

TABLE 2
Effervescent caffeine tablet ingredients

| Dry Ingredients | Weight % |
|---|---|
| mannitol | 25-40 |
| maltodextrin | 25-40 |
| sweetener | 0.1-0.5 |
| caffeine | 1-10 |
| tartaric acid | 4-6 |
| citric acid | 4-6 |
| sodium bicarbonate | 8-15 |
| sodium chloride | 0.1-0.5 |
| silicon dioxide | 0-2 |
| stearic acid | 0-2 |
| magnesium stearate | 0-2 |
| sodium stearyl fumarate | 0-2 |

### Example 2. Effervescent Tablet Containing L-Theanine, GABA, and Lemon Balm Extract

An effervescent tablet according to an embodiment of the disclosure was prepared according to Example 1, but including a mixture of L-theanine, GABA, and lemon balm extract as the active ingredient. The dry blend formulation is provided in Table 3.

TABLE 3
Effervescent L-theanine, GABA, and
Lemon Balm tablet ingredients

| Dry Ingredients | Weight % |
|---|---|
| isomalt | 22-33 |
| EMDEX ® | 30-45 |
| sweetener | 0.1-0.5 |
| L-theanine | 3-5 |
| GABA | 4-6 |
| lemon balm extract | 2-8 |
| tartaric acid | 4-6 |
| citric acid | 4-6 |
| sodium bicarbonate | 8-15 |
| sodium chloride | 0.1-0.5 |
| sodium stearyl fumarate | 0.5-1.5 |

### Example 3. Effervescent Tablet Containing Caffeine, Taurine, and Vitamin C

An effervescent tablet according to an embodiment of the disclosure was prepared according to Example 1, but including a mixture of caffeine, taurine, and vitamin C as the active ingredient. The dry blend formulation is provided in Table 4.

TABLE 4
Effervescent caffeine, taurine, and vitamin C tablet ingredients

| Dry Ingredients | Weight % |
|---|---|
| isomalt | 20-30 |
| EMDEX ® | 30-45 |
| sweetener | 0.1-0.5 |
| caffeine | 3-5 |
| taurine | 4-6 |
| vitamin C | 4-6 |
| sodium citrate | 2-3 |
| tartaric acid | 4-6 |
| citric acid | 4-6 |
| sodium bicarbonate | 8-15 |
| sodium chloride | 0.1-0.5 |
| sodium stearyl fumarate | 0.5-1.5 |

### Example 4. Effervescent Tablet Containing Caffeine, L-Theanine, Sunflower Lecithin, and Panax ginseng

An effervescent tablet according to an embodiment of the disclosure was prepared according to Example 1, but including a mixture of caffeine, L-theanine, sunflower lecithin, and Panax ginseng as the active ingredients. The dry blend formulation is provided in Table 5.

TABLE 5
Effervescent L-theanine, caffeine, sunflower lecithin,
and Panax ginseng tablet ingredients

| Dry Ingredients | Weight % |
|---|---|
| isomalt | 22-35 |
| EMDEX ® | 28-43 |
| sweetener | 0.1-0.5 |
| caffeine | 3-5 |
| L-theanine | 4-6 |
| sunflower lecithin | 0.5-1.5 |
| Panax ginseng | 0.4-0.6 |
| sodium citrate | 2-3 |
| tartaric acid | 4-6 |
| citric acid | 4-6 |
| sodium bicarbonate | 8-15 |
| sodium chloride | 0.1-0.5 |
| sodium stearyl fumarate | 0.5-1.5 |

### Example 5. Two-Layer Tablets

Multi-layer tablets according to an embodiment of the disclosure were prepared including nicotine as the active ingredient. One layer is effervescent, and provides a rapid release of nicotine. Another layer is non-effervescent and more gradually releases nicotine.

The effervescent composition was prepared from a dry blend formulation as provided in Table 6. The dry materials (EMDEX®, isomalt, sweetener, nicotine bitartrate, salt, pH adjuster, and a pre-formed mixture of sodium bicarbonate, citric acid, and tartaric acid) were each passed through an 18 mesh screen, then mixed in a V-blender until homogenous. Following the mixing period, flavorant and a lubricant (e.g., sodium stearyl fumarate) were added, followed by further mixing.

TABLE 6
Effervescent composition ingredients

| Dry Ingredients | Weight % |
|---|---|
| EMDEX ® | 40-55 |
| isomalt | 20-30 |
| sweetener | 0.4-0.6 |
| nicotine bitartrate | 0.7-1.1 |
| tartaric acid | 4-6 |
| citric acid | 4-6 |
| sodium bicarbonate | 8-15 |
| sodium chloride | 0.2-0.4 |
| pH adjuster | 2-3 |
| flavorant | 0.4-0.6 |
| sodium stearyl fumarate | 0-2 |

The non-effervescent composition was prepared from a dry blend formulation as provided in Table 7. The dry materials (EMDEX®, isomalt, sweetener, nicotine bitartrate, salt, and pH adjuster) were mixed in a V-blender until homogenous.

TABLE 7
Non-effervescent composition ingredients

| Dry Ingredients | Weight % |
|---|---|
| EMDEX ® | 40-60 |
| isomalt | 20-30 |
| hydroxypropylcellulose | 10-20 |
| sweetener | 0.4-0.6 |
| nicotine bitartrate | 0.7-1.1 |
| sodium chloride | 0.2-0.4 |
| pH adjuster | 2-3 |
| flavorant | 0.4-0.6 |
| sodium stearyl fumarate | 0.5-1.5 |
| Colorant | 0.5-1.5 |

Following the mixing period, flavorant, colorant, and a lubricant (e.g., sodium stearyl fumarate) were added, followed by further mixing. Layered tablets were prepared using a punch press, forming tablets weighing about 700 mg each. In a particular embodiment, the tablets are bi-layered, with each of the two layers containing approximately 350 mg of the respective compositions.

What is claimed is:

1. A multi-layered tablet configured for oral use, the tablet comprising a first layer comprising an effervescent composition and a second layer comprising a non-effervescent composition, wherein:
the effervescent composition comprises an effervescent material capable of causing effervescence in the oral cavity; one or more fillers in a total amount of at least about 30% by weight, based on the total weight of the effervescent composition, wherein the one or more fillers include at least one sugar alcohol; and a first active ingredient; and
the non-effervescent composition comprises one or more fillers in a total amount of at least about 30% by weight, based on the total weight of the non-effervescent composition, wherein the one or more fillers include at least one sugar alcohol; a binder; and a second active ingredient.

2. The multi-layered tablet of claim 1, wherein the first and second active ingredients are independently selected from the group consisting of nicotine components, botanical materials, stimulants, amino acids, vitamins, cannabinoids, cannabimimetics, terpenes, nutraceuticals, pharmaceutical agents, and combinations thereof.

3. The multi-layered tablet of claim 1, wherein the first active ingredient comprises a nicotine component.

4. The multi-layered tablet of claim 1, wherein the first and second active ingredients comprise a nicotine component.

5. The multi-layered tablet of claim 4, wherein the nicotine component is present in an amount from about 0.001 to about 10% by weight in each composition, calculated as the free base and based on the total weight of each composition.

6. The multi-layered tablet of claim 1, wherein the one or more fillers comprise isomalt, polysaccharides, or a combination thereof.

7. The multi-layered tablet of claim 1, wherein the one or more fillers comprise isomalt, glucose, and starch-derived polysaccharides.

8. The multi-layered tablet of claim 7, wherein the one or more fillers comprise from about 60% to about 90% by weight of each of the effervescent and the non-effervescent compositions, based on the total weight of each composition.

9. The multi-layered tablet of claim 1, wherein the particle size of the one or more fillers in the effervescent composition is less than about 35 microns.

10. The multi-layered tablet of claim 1, wherein the effervescent composition, the non-effervescent composition, or both, further comprise one or more additives selected from the group consisting of flavorants, sweeteners, tobacco materials, taste modifiers, salts, binders, buffering agents, colorants, humectants, oral care additives, preservatives, disintegration aids, antioxidants, flow aids, compressibility aids, and combinations thereof.

11. The multi-layered tablet of claim 10, wherein the particle size of any flavorants, sweeteners, taste modifiers, salts, binders, buffering agents, colorants, humectants, emulsifiers, oral care additives, preservatives, disintegration aids, antioxidants, flow aids, and compressibility aids which may be present in the effervescent composition is less than about 35 microns.

12. The multi-layered tablet of claim 1, wherein the binder is a cellulose derivative.

13. The multi-layered tablet of claim 12, wherein the cellulose derivative is a cellulose ether.

14. The multi-layered tablet of claim 13, wherein the cellulose ether is hydroxypropylcellulose.

15. The multi-layered tablet of claim 1, wherein the multi-layered tablet is substantially free of tobacco material.

16. A composition for use in an oral product comprising at least two layered portions, the composition comprising:
an effervescent portion comprising an effervescent material capable of causing effervescence in the oral cavity; one or more fillers in a total amount of at least about 30% by weight, based on the total weight of the effervescent portion, wherein the one or more fillers include at least one sugar alcohol; and a first active ingredient; and
a non-effervescent portion comprising one or more fillers in a total amount of at least about 30% by weight, based on the total weight of the non-effervescent portion, wherein the one or more fillers include at least one sugar alcohol; a binder; and a second active ingredient,
wherein the oral product is in the form of a tablet.
